(19) United States
(12) Patent Application Publication
(10) Pub. No.: US 2007/0105789 A1
(43) Pub. Date: May 10, 2007

(54) Combinatorial chemotherapy treatment using Na+/K+ ATPase inhibitors

(75) Inventors: Mehran Khodadoust, Brookline, MA (US); Ajay Sharma, Sudbury, MA (US); Reimar Bruening, Fremont, CA (US)

Correspondence Address: FISH & NEAVE IP GROUP;ROPES & GRAY LLP, ONE INTERNATIONAL PLACE, BOSTON, MA, 02110-2624, US

(73) Assignee: Bionaut Pharmaceuticals, Inc., Cambridge, MA (US)

(21) Appl. No.: 11/441,396

(22) Filed: May 24, 2006

Related U.S. Application Data
(63) Continuation-in-part of application No. 11/219,636, filed on Sep. 2, 2005.
(60) Provisional application No. 60/606,685, filed on Sep. 2, 2004.

(51) Int. Cl. A61K 31/70; A01N 43/04

(52) U.S. Cl. 514/34

(57) ABSTRACT

The reagent, pharmaceutical formulation, kit, and methods of the invention provides a new approach to alleviate or eliminate certain negative effects associated with the use of certain cancer treatment agents (e.g. chemotherapy therapeutics, etc.) or regimens (e.g. radio therapies, etc.), including stimulation of the hypoxic stress response in tumor cells. The reagent and pharmaceutical formulation of the invention relates to Na⁺/K⁺-ATPase inhibitors, such as cardiac glycosides, including bufadieneolides or their corresponding aglycones (e.g., proscillaridin, scillaren, and scillarenin, etc.), especially in oral formulations and/or solid dosage forms containing more than 1 mg of active ingredients.

### REFERENCE TO RELATED APPLICATION

This application is a continuation-in-part application of U.S. Ser. No. 11/219,636, filed on Sep. 2, 2005, which claims the benefit of the filing date of U.S. Provisional Application Ser. No. 60/606,685, entitled “COMBINATORIAL CHEMOTHERAPY TREATMENTS USING CARDIAC GLYCOSIDES AND OTHER Na+/K+-ATPASE INHIBITORS,” and filed on Sep. 2, 2004. The teachings of the referenced applications are incorporated herein by reference.

### BACKGROUND OF THE INVENTION

HIF-1 is a transcription factor and is critical to survival in hypoxic conditions, both in cancer and cardiac cells. HIF-1 is composed of the O₂⁻ and growth factor-regulated subunit HIF-1α, and the constitutively expressed HIF-1β subunit (arylhydrocarbon receptor nuclear translocator, ARNT), both of which belong to the basic helix-loop-helix (bHLH)-PAS (PER, ARNT, SIM) protein family. So far in the human genome 3 isoforms of the subunit of the transcription factor HIF have been identified: HIF-1, HIF-2 (also referred to as EPAS-1, MOP2, HLF, and HRF), and HIF-3 (of which HIF-32 also referred to as IPAS, inhibitory PAS domain).

Under normoxic conditions, HIF-1α is targeted to ubiquitinylation by pVHL and is rapidly degraded by the proteasome. This is triggered through posttranslational HIF-hydroxylation on specific proline residues (proline 402 and 564 in human HIF-1α protein) within the oxygen dependent degradation domain (ODDD), by specific HIF-prolyl hydroxylases (HPH1-3 also referred to as PHD1-3) in the presence of iron, oxygen, and 2-oxoglutarate. The hydroxylated protein is then recognized by pVHL, which functions as an E3 ubiquitin ligase. The interaction between HIF-1α and pVHL is further accelerated by acetylation of lysine residue 532 through an N-acetyltransferase (ARD I). Concurrently, hydroxylation of the asparagine residue 803 within the C-TAD also occurs by an asparaginyl hydroxylase (also referred to as FIH-1), which by its turn does not allow the coactivator p300/CBP to bind to HIF-1α subunit. In hypoxia HIF-1α remains not hydroxylated and stays away from interaction with pVHL and CBP/p300 (FIG. 6). Following hypoxic stabilization HIF-1α translocates to the nucleus where it hetero-dimerizes with HIF-1β. The resulting activated HIF-1 drives the transcription of over 60 genes important for adaptation and survival under hypoxia including glycolytic enzymes, glucose transporters Glut-1 and Glut-3, endothelin-1 (ET-1), VEGF (vascular endothelial growth factor), tyrosine hydroxylase, transferrin, and erythropoietin (Brahimi-Horn et al., 2001 Trends Cell Biol 11(11): S32-S36.; Beasley et al., 2002 Cancer Res 62(9): 2493-2497; Fukuda et al., 2002 J Biol Chem 277(41): 38205-38211; Maxwell and Ratcliffe, 2002 Semin Cell Dev Biol 13(1): 29-37).

It is an object of the present invention to improve the use of those an anti-cancer agent that induces a hypoxic stress response in tumor cells.

### SUMMARY OF THE INVENTION

The inventors have discovered that certain anti-tumor agents, in addition to their cancer-killing effects, in fact also promote a hypoxic stress response in tumor cells. The hypoxia stress response in turn promotes tumor growth, by promoting cell survival through its induction of angiogenesis and its activation of anaerobic metabolism, which have a direct negative consequence on clinical and prognostic parameters, and create a therapeutic challenge, including refractory cancer.

This hypoxic response includes induction of HIF-1-dependent transcription, which exerts complex effect on tumor growth, and involves the activation of several adaptive pathways.

Through the use of cellular assays that report a cells response to stress, the inventors have discovered for the first time that Na⁺/K⁺-ATPase inhibitors (such as the cardenolide cardiac glycoside Ouabain, and, to an even larger degree, the bufadienolide cardiac glycoside BNC-4 (i.e., Proscillaridin), and their respective aglycones) induce a signal that prevents cancer cells to respond to stresses such as hypoxic stress through transcriptional inhibition of Hypoxia Inducible Factor (HIF-1α) biosynthesis.

The inventors have discovered that the cellular and systemic responses share common endogenous cardiac glycosides, including ouabain and proscillaridin. However, the inventors also found that cardiac glycosides serve different roles in the cellular and systemic responses to hypoxic stress. Specifically, at the system level, cardiac glycosides are produced to mediate the body's response to hypoxic stress, including a role in regulating heart rate and increasing blood pressure associated with chronic hypoxic stress. Thus, endogenous cardiac glycosides' properties as mediators of such systemic response to hypoxia have been explored in the development of cardiovascular medications. Cardiac glycosides used in such medications, such as digoxin, ouabain and proscillaridin, are steroidal compounds chemically identical to endogenous cardiac glycosides.

In contrast, at the cellular level, cardiac glycosides inhibit a cell from making its normal survival response to hypoxic conditions, e.g., VEGF secretion, and theoretically enable the body to conserve limited resources so as to ensure the survival of the major organs. These findings demonstrate the existence of a cellular regulatory pathway that can modulate a cell's response to stress, the modulation of which cellular regulatory pathway may provide novel, effective treatment methods, such as the treatment of cancers. These findings also demonstrate a novel role for the systemic mediator of the body's response to hypoxic stress (e.g., the cardiac glycosides) in modulating normal cellular responses to hypoxia.

While not wishing to be bound by any particular theory, these Na⁺/K⁺-ATPase inhibitors at the cellular level bind to the sodium-potassium channel (Na⁺/K⁺-ATPase), and induces a signal that results in anti-proliferative events in cancer cells. This binding and signaling event proceeds independently from the pump-inhibition effect of these Na⁺/K⁺-ATPase inhibitors, and thus presents a novel mechanism for cancer treatment. Therefore, this discovery forms one basis for using cardiac glycosides (such as Proscillaridin, and their aglycones) in anti-cancer therapy. The anti-cancer therapy of the instant invention is useful in treating refractory cancers, especially those HIF-1α-associated refractory cancers.

Thus a salient feature of the present invention is the discovery that certain anti-tumor agents induce a hypoxic stress response in tumor cells, and that Na⁺/K⁺-ATPase inhibitors, such as cardiac glycosides, can be used to reduce that response and improve the efficacy of those anti-tumor agents.

One aspect of the invention provides a pharmaceutical formulation comprising a Na⁺/K⁺-ATPase inhibitor (such as a cardiac glycoside, and preferably in an oral dosage form), and an anti-cancer agent that induces a hypoxic stress response in tumor cells, formulated in a pharmaceutically acceptable excipient and suitable for use in humans to treat a neoplastic disorder.

Another aspect of the invention provides a kit for treating a patient having a neoplastic disorder, comprising a Na⁺/K⁺-ATPase inhibitor (such as a cardiac glycoside, and preferably in an oral dosage form) and an anti-cancer agent that induces a hypoxic stress response in tumor cells, each formulated in premeasured doses for conjoint administration to a patient.

Yet another aspect of the invention provides a method for treating a patient having a neoplastic disorder comprising administering to the patient an effective amount of a Na⁺/K⁺-ATPase inhibitor (such as a cardiac glycoside, and preferably in an oral dosage form) and an anti-cancer agent that induces a hypoxic stress response in tumor cells.

In a related aspect, the invention provides a use of a Na⁺/K⁺-ATPase inhibitor (such as a cardiac glycoside, and preferably in an oral dosage form) in the manufacture of a medicament in an oral dosage form, for treating a patient having a neoplastic disorder, said Na⁺/K⁺-ATPase inhibitor is administered with an anti-cancer agent that induces a hypoxic stress response in tumor cells.

Still another aspect of the invention provides a method for promoting treatment of patients having a neoplastic disorder, comprising packaging, labeling and/or marketing a Na⁺/K⁺-ATPase inhibitor (such as a cardiac glycoside, and preferably in an oral dosage form) to be used in conjoint therapy for treating a patient having a neoplastic disorder with an anti-cancer agent that induces a hypoxic stress response in tumor cells.

In a related aspect, the invention provides a use of a Na⁺/K⁺-ATPase inhibitor (such as a cardiac glycoside, and preferably in an oral dosage form) in the packaging, labeling and/or marketing of a medicament in an oral dosage form, for promoting treatment of patients having a neoplastic disorder, said Na⁺/K⁺-ATPase inhibitor is administered in conjoint therapy with an anti-cancer agent that induces a hypoxic stress response in tumor cells.

Another aspect of the invention relates to a method for promoting treatment of patients having a neoplastic disorder, comprising packaging, labeling and/or marketing an anti-cancer agent that induces a hypoxic stress response in tumor cells to be used in conjoint therapy with a Na⁺/K⁺-ATPase inhibitor (such as a cardiac glycoside, and preferably in an oral dosage form) for treating a patient having a neoplastic disorder.

In a related aspect, the invention provides a use of an anti-cancer agent that induces a hypoxic stress response in tumor cells in the packaging, labeling and/or marketing of a medicament, for promoting treatment of patients having a neoplastic disorder, said anti-cancer agent is administered in conjoint therapy with a Na⁺/K⁺-ATPase inhibitor (such as a cardiac glycoside, and preferably in an oral dosage form) in an oral dosage form.

For any of the aspects of the invention described herein, the following embodiments, each independent of one another as appropriate, and is able to combine with any of the other embodiment when appropriate, are contemplated below.

In certain preferred embodiments, the Na⁺/K⁺-ATPase inhibitor is a cardiac glycoside or aglycone thereof, such as a bufadienolide cardiac glycoside or aglycone thereof, preferably formulated in a pharmaceutically acceptable excipient and suitable for use in humans. The bufadienolide or aglycone thereof may be a solid oral dosage form of at least about 1.5 mg, about 2.0 mg, about 2.25 mg, about 2.5 mg, about 3.0 mg, about 4.0 mg, about 5.0 mg, about 7.5 mg, about 10 mg, or about 15 mg.

In certain embodiments, the cardiac glycoside, in combination with the anti-cancer agent, has an IC₅₀ for killing one or more different cancer cell lines that is at least 2 fold less relative to the IC₅₀ of the cardiac glycoside alone, and even more preferably at least 5, 10, 50 or even 100 fold less.

In certain embodiments, the cardiac glycoside, in combination with the anti-cancer agent, has an EC₅₀ for treating the neoplastic disorder that is at least 2 fold less relative to the EC₅₀ of the cardiac glycoside alone, and even more preferably at least 5, 10, 50 or even 100 fold less.

In certain embodiments, the cardiac glycoside has an IC₅₀ for killing one or more different cancer cell lines of 500 nM or less, and even more preferably 200 nM, 100 nM, 10 nM or even 1 nM or less.

In certain embodiments, the Na⁺/K⁺-ATPase inhibitor has a therapeutic index of at least about 2, preferably at least about 3, 5, 8, 10, 15, 20, 25, 30, 40, or about 50. Therapeutic index refers to the ratio between the minimum toxic serum concentration of a compound, and a therapeutically effective serum concentration sufficient to achieve a pre-determined therapeutic end point. For example, the therapeutic end point may be >50% or 60% inhibition of tumor growth (compared to an appropriate control) in a xenograph nude mice model, or in clinical trial.

In certain embodiments, the treatment period is about 1 month, 3 months, 6 months, 9 months, 1 year, 3 years, 5 years, 10 years, 15 years, 20 years, or the life-time of the individual.

In certain embodiments, the oral dosage form maintains an effective steady state serum concentration of about 10-100 ng/mL, about 15-80 ng/mL, about 20-50 ng/mL, or about 20-30 ng/mL.

In certain embodiments, the steady state serum concentration is reached by administering a total dose of about 5-10 mg/day, and a continuing dose(s) of about 1.5-5 mg/day in a human individual, preferably over the subsequent 1-3 days.

In certain embodiments, the oral dosage form comprises a total daily dose of about 1-7.5 mg, about 1.5-5 mg, or about 3-4.5 mg per human individual.

In certain embodiments, the oral dosage form is a solid oral dosage form.

In certain embodiments, the oral dosage form comprises a daily dose of 2-3 times of 1.5 mg cardiac glycoside or an aglycone thereof.

Unless otherwise indicated, the total daily dose may be administered as a single dose, or in as many doses as the physicians may choose.

In certain embodiments, the total daily dose may be administered as a single dose for, e.g., patient convenience, and/or better patient compliance.

In certain embodiments, the Cₘₐₓ is kept low by administering the total daily dosage over multiple doses (e.g., 2-5 doses, or 3 doses). This may be beneficial for patients who exibits certain side effects such as nausea and vomiting, for patients with weak heart muscles, or who otherwise do not tolerate relatively high doses or Cₘₐₓ well.

In certain embodiments, the oral dosage form comprise a single solid dose of about 1 mg, 1.5 mg, 2 mg, 2.5 mg, 3 mg, 3.5 mg, 4 mg, 4.5 mg, 5 mg, 5.5 mg, 6 mg, 6.5 mg, or about 7 mg of active ingredient.

In certain embodiments, the cardiac glycoside is represented by the general formula:

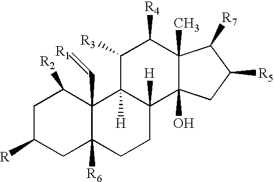

wherein

R represents a glycoside of 1 to 6 sugar residues, or —OH;

R₁ represents H,H; H,OH; or ═O;

R₂, R₃, R₄, R₅, and R₆ each independently represents hydrogen or —OH;

R₇ represents

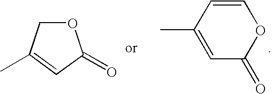

In certain preferred embodiments, the sugar residues are selected from L-rhamnose, D-glucose, D-digitoxose, D-digitalose, D-digginose, D-sarmentose, L-vallarose, and D-fructose. In certain embodiments, these sugars are in the β-conformation. The sugar residues may be acetylated, e.g., to effect the lipophilic character and the kinetics of the entire glycoside. In certain preferred embodiments, the glycoside is 1-4 sugar residues in length.

In certain embodiments, the cardiac glycoside comprises a steroid core with either a pyrone substituent at C17 (the “bufadienolides form”) or a butyrolactone substituent at C17 (the “cardenolide” form).

In certain embodiments, the cardiac glycoside is a bufadienolide comprising a steroid core with a pyrone substituent R7 at C17. The cardiac glycoside may have an IC₅₀ for killing one or more different cancer cell lines of about 500 nM, 200 nM, 100 nM, 10 nM or even 1 nM or less.

In certain embodiments, the cardiac glycoside is proscillaridin (e.g., Merck Index registry number 466-06-8) or scillaren (e.g., Merck Index registry number 11003-70-6).

In certain embodiments, the aglycone is scillarenin (e.g., Merck Index registry number 465-22-5).

In certain embodiments, the cardiac glycoside is selected from digitoxigenin, digoxin, lanatoside C, Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin, calotoxin, convallatoxin, oleandrigenin, bufalin, periplocyrnarin, digoxin (CP 4072), strophanthidin oxime, strophanthidin semicarbazone, strophanthidinic acid lactone acetate, ernicyrnarin, sannentoside D, sarverogenin, sarmentoside A, sarmentogenin, or a pharmaceutically acceptable salt, ester, amide, or prodrug thereof.

In certain preferred embodiments, the cardiac glycoside is ouabain or proscillaridin.

Other Na⁺/K⁺-ATPase inhibitors are available in the literature. See, for example, U.S. Pat. No. 5,240,714 which describes a non-digoxin-like Na⁺/K⁺-ATPase inhibitory factor. Recent evidence suggests the existence of several endogenous Na⁺/K⁺-ATPase inhibitors in mammals and animals. For instance, marinobufagenin (3,5-dihydroxy-14,15-epoxy bufodienolide) may be useful in the current combinatorial therapies.

Those skilled in the art can also rely on screening assays to identify compounds that have Na⁺/K⁺-ATPase inhibitory activity. PCT Publications WO00/44931 and W002/42842, for example, teach high-throughput screening assays for modulators of Na⁺/K⁺-ATPases.

The Na⁺/K⁺-ATPase consists of at least two dissimilar subunits, the large α subunit with all known catalytic functions and the smaller glycosylated β subunit with chaperonic function. In addition there may be a small regulatory, so-called FXYD-peptide. Four α peptide isoforms are known and isoform-specific differences in ATP, Na⁺ and K⁺ affinities and in Ca²⁺ sensitivity have been described. Thus changes in Na⁺/K⁺-ATPase isoform distribution in different tissues, as a function of age and development, electrolytes, hormonal conditions etc. may have important physiological implications. Cardiac glycosides like ouabain are specific inhibitors of the Na⁺/K⁺-ATPase. The four α peptide isoforms have similar high ouabain affinities with K_{d} of around 1 nM or less in almost all mammalian species. In certain embodiments, the Na⁺/K⁺-ATPase inhibitor is more selective for complexes expressed in non-cardiac tissue, relative to cardiac tissue.

In certain embodiments, the anti-cancer agent induces redox-sensitive transcription.

In certain embodiments, the anti-cancer agent induces HIF-1α-dependent transcription.

In certain embodiments, the anti-cancer agent induces expression of one or more of cyclin G2, IGF2, IGF-BP1, IGF-BP2, IGF-BP3, EGF, WAF-1, TGF-α, TGF-β3, ADM, EPO, IGF2, EG-VEGF, VEGF, NOS2, LEP, LRP1, HK1, HK2, AMF/GP1, ENO1, GLUT1, GAPDH, LDHA, PFKBF3, PKFL, MIC1, NIP3, NIX and/or RTP801.

In certain embodiments, the anti-cancer agent induces mitochondrial dysfunction and/or caspase activation.

In certain embodiments, the anti-cancer agent induces cell cycle arrest at G2/M in the absence of said cardiac glycoside.

In certain embodiments, the anti-cancer agent is an inhibitor of chromatin function.

In certain embodiments, the anti-cancer agent is a DNA topoisomerase inhibitor, such as selected from adriamycin, amsacrine, camptothecin, daunorubicin, dactinomycin, doxorubicin, eniposide, epirubicin, etoposide, idarubicin, irinotecan (CPT-11) and mitoxantrone.

In certain embodiments, the anti-cancer agent is a microtubule inhibiting drug, such as a taxane, including paclitaxel, docetaxel, vincristin, vinblastin, nocodazole, epothilones and navelbine.

In certain embodiments, the anti-cancer agent is a DNA damaging agent, such as actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16).

In certain embodiments, the anti-cancer agent is an antimetabolite, such as a folate antagonists, or a nucleoside analog. Exemplary nucleoside analogs include pyrimidine analogs, such as 5-fluorouracil; cytosine arabinoside, and azacitidine. In other embodiments, the nucleoside analog is a purine analog, such as 6-mercaptopurine; azathioprine; 5-iodo-2′-deoxyuridine; 6-thioguanine; 2-deoxycoformycin, cladribine, cytarabine, fludarabine, mercaptopurine, thioguanine, and pentostatin. In certain embodiments, the nucleoside analog is selected from AZT (zidovudine); ACV; valacylovir; famiciclovir; acyclovir; cidofovir; penciclovir; ganciclovir; Ribavirin; ddC; ddl (zalcitabine); lamuvidine; Abacavir; Adefovir; Didanosine; d4T (stavudine); 3TC; BW 1592; PMEA/bis-POM PMEA; ddT, HPMPC, HPMPG, HPMPA, PMEA, PMEG, dOTC; DAPD; Ara-AC, pentostatin; dihydro-5-azacytidine; tiazofurin; sangivamycin; Ara-A (vidarabine); 6-MMPR; 5-FUDR (floxuridine); cytarabine (Ara-C; cytosine arabinoside); 5-azacytidine (azacitidine); HBG [9-(4-hydroxybutyl)guanine], (1S,4R)-4-[2-amino-6-cyclopropyl-amino)-9H-purin-9-yl]-2-cyclopentene-1-methanol succinate (“159U89“), uridine; thymidine; idoxuridine; 3-deazauridine; cyclocytidine; dihydro-5-azacytidine; triciribine, ribavirin, and fludrabine.

In certain embodiments, the nucleoside analog is a phosphate ester selected from the group consisting of: Acyclovir; 1-β-D-arabinofuranosyl-E-5-(2-bromovinyl)uracil; 2′-fluorocarbocyclic-2′-deoxyguanosine; 6′-fluorocarbocyclic-2′-deoxyguanosine; 1-(β-D-arabinofuranosyl)-5(E)-(2-iodovinyl)uracil; {(1r-1α,2β,3α)-2-amino-9-(2,3-bis(hydroxymethyl)cyclobutyl)-6H-purin-6-one}Lobucavir; 9H-purin-2-amine, 9-((2-(1-methylethoxy)-1-((1-methylethoxy)methyl)ethoxy)methyl)-(9Cl); trifluorothymidine; 9->(1,3-dihydroxy-2-propoxy)methylguanine (ganciclovir); 5-ethyl-2′-deoxyuridine; E-5-(2-bromovinyl)-2′-deoxyuridine; 5-(2-chloroethyl)-2′-deoxyuridine; buciclovir; 6-deoxyacyclovir; 9-(4-hydroxy-3-hydroxymethylbut-1-yl)guanine; E-5-(2-iodovinyl)-2′-deoxyuridine; 5-vinyl-1-β-D-arabinofuranosyluracil; 1-β-D-arabinofuranosylthymine; 2′-nor-2′deoxyguanosine; and 1-β-D-arabinofuranosyladenine.

In certain embodiments, the nucleoside analog modulates intracellular CTP and/or dCTP metabolism.

In certain preferred embodiments, the nucleoside analog is gemcitabine (GEMZAR®).

In certain embodiments, the anti-cancer agent is a DNA synthesis inhibitor, such as a thymidilate synthase inhibitors (such as 5-fluorouracil), a dihydrofolate reductase inhibitor (such as methoxtrexate), or a DNA polymerase inhibitor (such as fludarabine).

In certain embodiments, the anti-cancer agent is a DNA binding agent, such as an intercalating agent.

In certain embodiments, the anti-cancer agent is a DNA repair inhibitor.

In certain embodiments, the anti-cancer agent is part of a combinatorial therapy selected from ABV, ABVD, AC (Breast), AC (Sarcoma), AC (Neuroblastoma), ACE, ACe, AD, AP, ARAC-DNR, B-CAVe, BCVPP, BEACOPP, BEP, BIP, BOMP, CA, CABO, CAF, CAL-G, CAMP, CAP, CaT, CAV, CAVE ADD, CA-VP16, CC, CDDP/VP-16, CEF, CEPP(B), CEV, CF, CHAP, ChlVPP, CHOP, CHOP-BLEO, CISCA, CLD-BOMP, CMF, CMFP, CMFVP, CMV, CNF, CNOP, COB, CODE, COMLA, COMP, Cooper Regimen, COP, COPE, COPP, CP -Chronic Lymphocytic Leukemia, CP-Ovarian Cancer, CT, CVD, CVI, CVP, CVPP, CYVADIC, DA, DAT, DAV, DCT, DHAP, DI, DTIC/Tamoxifen, DVP, EAP, EC, EFP, ELF, EMA 86, EP, EVA, FAC, FAM, FAMTX, FAP, F-CL, FEC, FED, FL, FZ, HDMTX, Hexa-CAF, ICE-T, IDMTX/6-MP, IE, IfoVP, IPA, M-2, MAC-III, MACC, MACOP-B, MAID, m-BACOD, MBC, MC, MF, MICE, MINE, mini-BEAM, MOBP, MOP, MOPP, MOPP/ABV, MP—multiple myeloma, MP—prostate cancer, MTX/6-MO, MTX/6-MP/VP, MTX-CDDPAdr, MV—breast cancer, MV—acute myelocytic leukemia, M-VAC Methotrexate, MVP Mitomycin, MVPP, NFL, NOVP, OPA, OPPA, PAC, PAC-I, PA-Cl, PC, PCV, PE, PFL, POC, ProMACE, ProMACE/cytaBOM, PRoMACE/MOPP, Pt/VM, PVA, PVB, PVDA, SMF, TAD, TCF, TIP, TTT, Topo/CTX, VAB-6, VAC, VACAdr, VAD, VATH, VBAP, VBCMP, VC, VCAP, VD, VelP, VIP, VM, VMCP, VP, V-TAD, 5+2,7+3, “8 in 1.”

In certain embodiments, the anti-cancer agent is selected from altretamine, aminoglutethimide, amsacrine, anastrozole, asparaginase, bcg, bicalutamide, bleomycin, buserelin, busulfan, calcium folinate, campothecin, capecitabine, carboplatin, carmustine, chlorambucil, cisplatin, cladribine, clodronate, colchicine, crisantaspase, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

In certain embodiments, the anti-cancer agent is selected from tamoxifen, 4-(3-chloro-4-fluorophenylamino)-7-methoxy-6-(3-(4-α-morpholinyl)propoxy)quinazoline, 4-(3-ethynylphenylamino)-6,7-bis(2-methoxyethoxy)quinazoline, hormones, steroids, steroid synthetic analogs, 17α-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, chlorotrianisene, hydroxyprogesterone, aminoglutethimide, estramustine, medroxyprogesteroneacetate, leuprolide, flutamide, toremifene, Zoladex, antiangiogenics, matrix metalloproteinase inhibitors, VEGF inhibitors, ZD6474, SU6668, SU11248, anti-Her-2 antibodies (ZD1839 and OS1774), EGFR inhibitors, EKB-569, Imclone antibody C225, src inhibitors, bicalutamide, epidermal growth factor inhibitors, Her-2 inhibitors, MEK-1 kinase inhibitors, MAPK kinase inhibitors, P13 inhibitors, PDGF inhibitors, combretastatins, MET kinase inhibitors, MAP kinase inhibitors, inhibitors of non-receptor and receptor tyrosine kinases (imatinib), inhibitors of integrin signaling, and inhibitors of insulin-like growth factor receptors.

In certain embodiments, the anti-cancer agent is selected from an EGF-receptor antagonist, and arsenic sulfide, adriamycin, cisplatin, carboplatin, cimetidine, carminomycin, mechlorethamine hydrochloride, pentamethylmelamine, thiotepa, teniposide, cyclophosphamide, chlorambucil, demethoxyhypocrellin A, melphalan, ifosfamide, trofosfamide, Treosulfan, podophyllotoxin or podophyllotoxin derivatives, etoposide phosphate, teniposide, etoposide, leurosidine, leurosine, vindesine, 9-aminocamptothecin, camptoirinotecan, crisnatol, Chloroambucil, megestrol, methopterin, mitomycin C, ecteinascidin 743, busulfan, carmustine (BCNU), lomustine (CCNU), lovastatin, 1-methyl-4-phenylpyridinium ion, semustine, staurosporine, streptozocin, thiotepa, phthalocyanine, dacarbazine, aminopterin, methotrexate, trimetrexate, thioguanine, mercaptopurine, fludarabine, pentastatin, cladribin, cytarabine (ara C), porfiromycin, 5-fluorouracil, 6-mercaptopurine, doxorubicin hydrochloride, leucovorin, mycophenoloc acid, daunorubicin, deferoxamine, floxuridine, doxifluridine, ratitrexed, idarubicin, epirubican, pirarubican, zorubicin, mitoxantrone, bleomycin sulfate, mitomycin C, actinomycin D, safracins, saframycins, quinocarcins, discodermolides, vincristine, vinblastine, vinorelbine tartrate, vertoporfin, paclitaxel, tamoxifen, raloxifene, tiazofuran, thioguanine, ribavirin, EICAR, estramustine, estramustine phosphate sodium, flutamide, bicalutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethosone, gemcitabine hydrochloride, verapamil, VP-16, altretamine, thapsigargin, and topotecan.

In certain embodiments, the subject combinations are used to inhibit growth of a tumor cell selected from a pancreatic tumor cell, lung tumor cell, a prostate tumor cell, a breast tumor cell, a colon tumor cell, a liver tumor cell, a brain tumor cell, a kidney tumor cell, a skin tumor cell, an ovarian tumor cell and a leukemic blood cell.

In certain embodiments, the subject combination is used in the treatment of a proliferative disorder selected from renal cell cancer, Kaposi's sarcoma, chronic lymphocytic leukemia, lymphoma, mesothelioma, breast cancer, sarcoma, ovarian carcinoma, rectal cancer, throat cancer, melanoma, colon cancer, bladder cancer, mastocytoma, lung cancer, liver cancer, mammary adenocarcinoma, pharyngeal squamous cell carcinoma, prostate cancer, pancreatic cancer, gastrointestinal cancer, and stomach cancer.

In certain embodiments, the subject combination is used in the treatment of a solid tumor, such as a tumor in the pancreas, lung, kidney, ovarian, breast, prostate, gastric, colon, bladder, prostate, brain, skin, testicles, cervix, or liver.

In certain embodiments, the subject combination is used in the treatment of a hematological cancer.

It is contemplated that all embodiments of the invention may be combined with any other embodiment(s) of the invention.

### BRIEF DESCRIPTION OF THE DRAWINGS

FIG. 1. Schematic diagram of using Sentinel Line promoter-less trap vectors to generate active genetic sites expressing drug selection markers and/or reporters.

FIG. 2. Schematic diagram of creating a Sentinel Line by sequential isolation of cells resistant to positive and negative selection drugs.

FIG. 3. Adaptation of a cancer cell to hypoxia, which leads to activation of multiple survival factors. The HIF family acts as a master switch transcriptionally activating many genes and enabling factors necessary for glycolytic energy metabolism, angiogenesis, cell survival and proliferation, and erythropoiesis. The level of HIF proteins present in the cell is regulated by the rate of their synthesis in response to factors such as hypoxia, growth factors, androgens and others. Degradation of HIF depends in part on levels of reactive oxygen species (ROS) in the cell. ROS leads to ubiquitylation and degradation of HIF.

FIG. 4. FACS Analysis of Sentinel Lines. Sentinel Lines were developed by transfecting A549 (NSCLC lung cancer) and Panc-l (pancreatic cancer) cell lines with gene-trap vectors containing E. coli LacZ-encoded β-galactosidase (β-gal) as the reporter gene. The β-gal activity in Sentinel Lines (green) was measured by flow cytometry using a fluorogenic substrate fluoresescein di-beta-D-galactopyranoside (FDG). The auto-fluorescence of untransfected control cells is shown in purple. The graphs indicate frequency of cells (y-axis) and intensity of fluorescence (x-axis) in log scale. The bar charts on the right depict median fluorescent units of the FACS curves. They indicate a high level of reporter activity at the targeted site.

FIG. 5. Western Blot analysis of HIF-1α expression indicates that cardiac glycoside compounds inhibit HIF-1α expression.

FIG. 6. Demonstrates that BNC-1 inhibits HIF-1α synthesis.

FIG. 7. Demonstrates that BNC-1 induces ROS production and inhibits HIF-1α induction in tumor cells.

FIG. 8. Demonstrates that the cardiac glycoside compounds BNC-1 and BNC-4 directly or indirectly inhibits in tumor cells the secretion of the angiogenesis factor VEGF.

FIG. 9. These four charts show FACS analysis of response of a NSCLC Sentinel Line (A549), when treated 40 hrs with four indicated agents. Control (untreated) is shown in purple. Arrow pointing to the right indicates increase in reporter activity whereas inhibitory effect is indicated by arrow pointing to the left. The results indicate that standard chemotherapy drugs turn on survival response in tumor cells.

FIG. 10. Effect of BNC-4 on Gem citabine-induced stress responses visualized by A549 Sentinel Lines™.

FIG. 11. Pharmacokinetic analysis of BNC-1 delivered by osmotic pumps. Osmotic pumps (Model 2002, Alzet Inc) containing 200 μl of BNC-1 at 50, 30 or 20 mg/ml in 50% DMSO were implanted subcutaneously into nude mice. Mice were sacrificed after 24, 48 or 168 hrs, and plasma was extracted and analyzed for BNC-1 by LC-MS. The values shown are average of 3 animals per point.

FIG. 12. Shows effect of BNC-1 alone or in combination with standard chemotherapy on growth of xenografted human pancreatic tumors in nude mice.

FIG. 13. Shows anti-tumor activity of BNC-1 and Cytoxan against Caki-1 human renal cancer xenograft.

FIG. 14. Shows anti-tumor activity of BNC-1 alone or in combination with Carboplatin in A549 human non-small-cell-lung carcinoma.

FIG. 15. Titration of BNC-1 to determine minimum effective dose effective against Panc-1 human pancreatic xenograft in nude mice. BNC-1 (sc, osmotic pumps) was tested at 10, 5 and 2 mg/ml.

FIG. 16. Combination of BNC-1 with Gemcitabine is more effective than either drug alone against Panc-1 xenografts.

FIG. 17. Combination of BNC-1 with 5-FU is more effective than either drug alone against Panc-1 xenografts.

FIG. 18. Comparison of BNC-1 and BNC-4 in inhibiting hypoxia-mediated HIF-1α induction in human tumor cells (Hep3B cells).

FIG. 19. Comparison of BNC-1 and BNC-4 in inhibiting hypoxia-mediated HIF-1α induction in human tumor cells (Caki-1 and Panc-1 cells).

FIG. 20. BNC-4 blocks HIF-1α induction by a prolyl-hydroxylase inhibitor under normoxia.

FIG. 21. Results showing that the Bufadienolides are more potent Na⁺/K⁺-ATPase inhibitors and cell proliferation inhibitors than the Cardenolides.

FIG. 22. Results showing that BNC-4 alone can significantly reduce tumor growth in xenografted Panc-1 tumors in nude mice.

FIG. 23. Results showing pharmacokinetic analysis of BNC-4 delivered by osmotic pump, and that BNC-4 alone can significantly reduce tumor growth in xenografted Caki-1 human renal tumors in nude mice.

### DETAILED DESCRIPTION OF THE INVENTION

### I. Overview

The present invention is based in part on the discovery that certain anti-tumor agents in fact promote a hypoxic stress response in tumor cells. For instance, such anti-cancer agents induce expression of one or more of cyclin G2, IGF2, IGF-BP 1, IGF-BP2, IGF-BP3, EGF, WAF-1, TGF-α, TGF-β3, ADM, EPO, IGF2, EG-VEGF, VEGF, NOS2, LEP, LRP1, HK1, HK2, AMF/GP1, ENO1, GLUTI, GAPDH, LDHA, PFKBF3, PKFL, MICI, NIP3, NIX and/or RTP801. By promoting cell survival through its induction of angiogenesis and its activation of anaerobic metabolism, it is believed that the activation of a hypoxic stress response would be counteractive to the other anti-cancer activities of these drugs. A salient feature of the present invention is the discovery that Na⁺/K⁺-ATPase inhibitors (e.g. cardiac glycosides, including bufadienolides or cardenolides) can be used to reduce the induced hypoxic stress response and improve the efficacy of those anti-tumor agents.

In a preferred embodiment, the Na⁺/K⁺-ATPase inhibitors are formulated as oral dosage forms, for either single dose or multiple doses per day administration to patients in need thereof.

### II. Definitions

As used herein the term “animal” refers to mammals, preferably mammals such as humans. Likewise, a “patient” or “subject” to be treated by the method of the invention can mean either a human or non-human animal.

As used herein, the term “cancer” refers to any neoplastic disorder, including such cellular disorders as, for example, renal cell cancer, Kaposi's sarcoma, chronic leukemia, prostate cancer, breast cancer, sarcoma, pancreatic cancer, ovarian carcinoma, rectal cancer, throat cancer, melanoma, colon cancer, bladder cancer, mastocytoma, lung cancer, mammary adenocarcinoma, myeloma, lymphoma, pharyngeal squamous cell carcinoma, and gastrointestinal or stomach cancer. Preferably, the cancer which is treated in the present invention is melanoma, lung cancer, breast cancer, pancreatic cancer, prostate cancer, colon cancer, or ovarian cancer.

The “growth state” of a cell refers to the rate of proliferation of the cell and the state of differentiation of the cell.

As used herein, “hyper-proliferative disease” or “hyper-proliferative disorder” refers to any disorder which is caused by or is manifested by unwanted proliferation of cells in a patient. Hyper-proliferative disorders include but are not limited to cancer, psoriasis, rheumatoid arthritis, lamellar ichthyosis, epidermolytic hyperkeratosis, restenosis, endometriosis, and abnormal wound healing.

As used herein, “proliferating” and “proliferation” refer to cells undergoing mitosis.

As used herein, “unwanted proliferation” means cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyper-proliferative disease” or “hyper-proliferative disorder.”

As used herein, “transformed cells” refers to cells that have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control. For purposes of this invention, the terms “transformed phenotype of malignant mammalian cells” and “transformed phenotype” are intended to encompass, but not be limited to, any of the following phenotypic traits associated with cellular transformation of mammalian cells: immortalization, morphological or growth transformation, and tumorigenicity, as detected by prolonged growth in cell culture, growth in semi-solid media, or tumorigenic growth in immuno-incompetent or syngeneic animals.

### III. Exemplary Embodiments

Many Na⁺/K⁺-ATPase inhibitors are available in the literature. See, for example, U.S. Pat. No. 5,240,714 which describes a non-digoxin-like Na⁺/K⁺-ATPase inhibitory factor. Recent evidence suggests the existence of several endogenous Na⁺/K⁺-ATPase inhibitors in mammals and animals. For instance, marinobufagenin (3,5-dihydroxy-14,15-epoxy bufodienolide) may be useful in the current combinatorial therapies.

Those skilled in the art can also rely on screening assays to identify compounds that have Na⁺/K⁺-ATPase inhibitory activity. PCT Publications WO00/4493 1 and WO02/42842, for example, teach high-throughput screening assays for modulators of Na⁺/K⁺-ATPase.

The Na⁺/K⁺-ATPase consists of at least two dissimilar subunits, the large a subunit with all known catalytic functions and the smaller glycosylated b subunit with chaperonic function. In addition there may be a small regulatory, so-called FXYD-peptide. Four a peptide isoforms are known and isoform-specific differences in ATP, Na⁺ and K⁺ affinities and in Ca²⁺ sensitivity have been described. The alpha 1 isoform has been shown to be ubiquitously expressed in all cell types, while the alpha 2 and alpha 3 isoforms are expressed in more excitable tissues such as heart, muscle and CNS. Thus changes in Na⁺/K⁺-ATPase isoform distribution in different tissues, as a function of age and development, electrolytes, hormonal conditions etc. may have important physiological implications. Cardiac glycosides like ouabain are specific inhibitors of the Na⁺/K⁺-ATPase. The four a peptide isoforms have similar high ouabain affinities with K_{d} of around 1 nM or less in almost all mammalian species. In certain embodiments, the Na⁺/K⁺-ATPase inhibitor is more selective for complexes expressed in non-cardiac tissue, relative to cardiac tissue. The following section describes a preferred embodiments of Na⁺/K⁺-ATPase inhibitors—cardiac glycosides.

A. Exemplary Cardiac Glycosides

The inventors have demonstrated that Na⁺/K⁺-ATPase inhibitors (e.g. cardiac glycosides) are effective in suppressing hypoxia-induced gene expression, such as in cancer cells. For example, Na⁺/K⁺-ATPase inhibitors (e.g. cardiac glycosides) are effective in suppressing EGF, insulin and/or IGF-responsive gene expression in various growth factor responsive cancer cell lines. As another example, the inventors have observed that Na⁺/K⁺-ATPase inhibitors (e.g. cardiac glycosides) are effective in suppressing HIF-responsive gene expression in cancer cell lines and furthermore, Na⁺/K⁺-ATPase inhibitors (e.g. cardiac glycosides) are shown to have potent anti-proliferative effects in cancer cell lines.

The term “cardiac glycoside” or “cardiac steroid” is used in the medical field to refer to a category of compounds tending to have positive inotropic effects on the heart. As a general class of compounds, cardiac glycosides comprise a steroid core with either a pyrone or butenolide substituent at C17 (the “pyrone form” and “butenolide form”). Additionally, cardiac glycosides may optionally be glycosylated at C3. The form of cardiac glycosides without glycosylation is also known as “aglycone.” Most cardiac glycosides include one to four sugars attached to the 3β-OH group. The sugars most commonly used include L-rhamnose, D-glucose, D-digitoxose, D-digitalose, D-digginose, D-sarmentose, L-vallarose, and D-fructose. In general, the sugars affect the pharmacokinetics of a cardiac glycoside with little other effect on biological activity. For this reason, aglycone forms of cardiac glycosides are available and are intended to be encompassed by the term “cardiac glycoside” as used herein. The pharmacokinetics of a cardiac glycoside may be adjusted by adjusting the hydrophobicity of the molecule, with increasing hydrophobicity tending to result in greater absorption and an increased half-life. Sugar moieties may be modified with one or more groups, such as an acetyl group.

A large number of cardiac glycosides are known in the art for the purpose of treating cardiovascular disorders. Given the significant number of cardiac glycosides that have proven to have anticancer effects in the assays disclosed herein, it is expected that most or all of the cardiac glycosides used for the treatment of cardiovascular disorders may also be used for treating proliferative disorders. Examples of preferred cardiac glycosides include ouabain, digitoxigenin, digoxin and lanatoside C. Additional examples of cardiac glycosides include: Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin and calotoxin. Cardiac glycosides may be evaluated for effectiveness in the treatment of cancer by a variety of methods, including, for example: evaluating the effects of a cardiac glycoside on expression of a HIF-responsive gene in a cancer cell line or evaluating the effects of a cardiac glycoside on cancer cell proliferation.

Notably, cardiac glycosides affect proliferation of cancer cell lines at a concentration well below the known toxicity level. The IC₅₀ measured for ouabain across several different cancer cell lines ranged from about 15 nM to about 600 nM, or 8OnM to about 300 nM. The concentration at which a cardiac glycoside is effective as part of an anti-proliferative treatment may be further decreased by combination with an additional agent that negatively regulates HIF-responsive genes, such as a redox effector or a steroid signal modulator. For example, as shown herein, the concentration at which a cardiac glycoside (e.g. ouabain or proscillaridin) is effective for inhibiting proliferation of cancer cells is decreased 5-fold by combination with a steroid signal modulator (Casodex). Therefore, in certain embodiments, the invention provides combination therapies of cardiac glycosides with, for example, steroid signal modulators and/or redox effectors. Additionally, cardiac glycosides may be combined with radiation therapy, taking advantage of the radiosensitizing effect that many cardiac glycosides have.

One exemplary cardiac glycoside is proscillaridin, and its corresponding aglycone is scillarenin. Other cardiac glycosides, such as scillaren, may differ only in glycosylation from proscillaridin, and thus have the same aglycone.

Proscillaridin (BNC-4) is a natural product from the Squill plant, Urginea (=Scilla) maritima of the Liliaceae family, a.k.a., “Sea Onion.” The plant was used since antiquity against dropsy (Papyrus Ebers, 1554 B.C., see Jarcho S 1974, and Stannard J 1974, and historic references cited therein), presumably for its diuretic properties, and is thus one of the oldest drugs in medicine. Toad toxins, whose chemical structure is very similar to that of Proscillaridin, have been used in China under the name of Ch'an Su for several thousand years for similar indications.

Proscillaridin belongs to the class of cardiac glycosides, steroid-like natural products with a characteristic unsaturated lactone ring attached in beta configuration to carbon 17 (C17). Depending on the ring size, one distinguishes cardenolides (5-membered lactone ring with one double bond) and bufadienolides (6-membered lactone ring with two double bonds). Proscillaridin belongs to the bufadienolide group, while the more frequently used glycosides from the Digitalis plant (Digitoxin, Digoxin) are cardenolides.

On carbon 3 (C3), cardiac glycosides carry up to four sugar molecules, of which glucose and rhamnose are the most common (Proscillaridin is a 3-beta rhamnoside). Unlike in the majority of steroids, the junction between the C and D rings is cis in cardiac glycosides. This configuration, as well as an extended, conjugated δ-electronic system with an electron-withdrawing (δ⁻) terminus on carbon 17 in beta-configuration, seems to be essential for the cardiac activity of these compounds (see Thomas R, Gray P, Andrews J. 1990).

Botanical sources of proscillaridin are well-known in the art. For example, such information can be found at various websites, such as maltawildplants dot com/LILI/Urginea_maritima.html#BOT. The website shows that the concentration of proscillaridin in the dried squill bulb is about 500 ppm, but its close relative, scillaren, is about 10-times more at 6000 ppm. Although these two compounds slightly differ by the sugar side chains, they both have the same aglycone—scillarenin. As a result, one needs only about 1/10 as much raw material to produce a gram of scillarenin as one needs to produce an equal amount of proscillaridin.

According to the invention, the subject compositions (including the Na⁺/K⁺-ATPase inhibitors, e.g., the cardiac glycosides, the bufadienolides, proscillaridin etc.), are preferably formulated in oral dosage forms. The oral dosage forms of the composition may be in a single dose or multi-dose formulation. The single dose form may be better than the multi-dose form in terms of patient compliance, while the multi-dose form may be better than the single dose in terms of avoiding temporary over-dose due to the rapid absorption of certain subject compositions.

The multi-dose formula may comprise 2-3, or 2-4 doses per day, either in equal amounts, or adjusted for different doses for a particular dose (e.g., the first dose in the morning or the last dose before sleep may be a higher dose to compensate for the long intermission over night).

In certain embodiments, the subject Na⁺/K⁺-ATPase inhibitor is proscillaridin. Exemplary dosages of proscillaridin for the subject invention are provided below. The dosages of any other Na⁺/K⁺-ATPase inhibitors may be deduced based on the exemplary proscillaridin doses, taking into consideration their relative effectiveness and toxicity compared to those of proscillaridin.

In certain embodiments, the oral dosage form of proscillaridin, when delivered to an average adult human, achieves and maintains an effective steady state serum concentration of about 10-700 ng/mL, about 30-500 ng/mL, about 40-500 ng/mL, about 50-500 ng/mL, about 50-400 ng/mL, about 50-300 ng/mL, about 50-200 ng/mL, or about 50-100 ng/mL.

In certain embodiments, the lower end of the concentration is about 10-70 ng/mL, about 30-60 ng/mL, or about 40-50 ng/mL.

In certain embodiments, the high end of the concentration is about 70-500 ng/mL, about 100-500 ng/mL, about 300-500 ng/mL, or about 400-500 ng/mL.

To achieve a steady state level of about 50 ng/mL, a daily total dose of about 2-3 mg is administered to the average human patient. Anti-tumor activity of proscillaridin was observed at a steady state serum level of about 50 ng/mL in a xenograft nude mouse model, where greater than 60% TGI (tumor growth inhibition) was observed. Meanwhile, the maximum toxic dose (MTD) in mice corresponds to a serum levels of about 518 (±121) ng/ml of proscillaridin.

Thus in certain embodiments, about 3-10 mg, about 2.25-7.5 mg, about 1-7.5 mg, about 1.5-5 mg, or about 3-5 mg of proscillaridin is administered per day. In certain other embodiments, an initial dose of about 5-10 mg is administered in the first day, and about 1.5-5 mg is administered every day thereafter.

In certain embodiments, the oral dosage form comprises a daily dose of 2-3 times of 1.5 mg cardiac glycoside or an aglycone thereof.

B. Exemplary Anti-Cancer Agents

Pharmaceutical agents that may be used in the subject combination therapy with Na⁺/K⁺-ATPase inhibitors (e.g. cardiac glycosides) include, merely to illustrate: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These anti-cancer agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); anti-proliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, docetaxel, doxorubicin, epirubicin, hexamethylmelamineoxaliplatin, iphosphamide, melphalan, merchlorethamine, mitomycin, mitoxantrone, nitrosourea, paclitaxel, plicamycin, procarbazine, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes—dacarbazinine (DTIC); anti-proliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; chromatin disruptors.

These anti-cancer agents are used by itself with an HIF inhibitor, or in combination. Many combinatorial therapies have been developed in prior art, including but not limited to those listed in Table 1.

TABLE 1
Exemplary conventional combination cancer chemotherapy

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, Procarbazine, Prednisone |
| BEACOPP | Bleomycin, Etoposide, Doxorubicin, Cyclophosphamide, Vincristine, |
| | Procarbazine, Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, Prednisone, |
| | Asparaginase |
| CAMP | Cyclophosphamide, Doxorubicin, Methotrexate, Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or without/ |
| | Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, Doxorubicin, |
| | Cisplatin |
| ChlVPP | Chlorambucil, Vinblastine, Procarbazine, Prednisone |
| CHOP | Cyclophosphamide, Doxorubicin, Vincristine, Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, Prednisone |
| CMFVP | Cyclophosphamide, Methotrexate, Fluorouracil, Vincristine, |
| | Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, Leucovorin, Cytarabine |
| COMP | Cyclophosphamide, Vincristine, Methotrexate, Prednisone |
| Cooper Regimen | Cyclophosphamide, Methotrexate, Fluorouracil, Vincristine, |
| | Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, Prednisone |
| CP (Chronic | Chlorambucil, Prednisone |
| lymphocytic | |
| leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, Fluorouracil |
| ICE-T | Ifosfamide, Carboplatin, Etoposide, Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, Prednisone, Melphalan |
| MAC-III | Methotrexate, Leucovorin, Dactinomycin, Cyclophosphamide |
| MACC | Methotrexate, Doxorubicin, Cyclophosphamide, Lomustine |
| MACOP-B | Methotrexate, Leucovorin, Doxorubicin, Cyclophosphamide, |
| | Vincristine, Bleomycin, Prednisone |
| MAID | Mesna, Doxorubicin, Ifosfamide, Dacarbazine |
| m-BACOD | Bleomycin, Doxorubicin, Cyclophosphamide, Vincristine, |
| | Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, Prednisone, Doxorubicin, |
| | Bleomycin, Vinblastine |
| MP (multiple | Melphalan, Prednisone |
| myeloma) | |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute | Mitoxantrone, Etoposide |
| myelocytic | |
| leukemia) | |
| M-VAC | Vinblastine, Doxorubicin, Cisplatin |
| Methotrexate | |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, Etoposide, Cytarabine, |
| | Bleomycin, Vincristine, Methotrexate, Leucovorin, Cotrimoxazole |
| PRoMACE/MOPP | Prednisone, Doxorubicin, Cyclophosphamide, Etoposide, |
| | Mechlorethamine, Vincristine, Procarbazine, Methotrexate, Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, Vincristine, Bleomycin, |
| | Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, Cisplatin, Bleomycin |
| VAC | Vincristine, Dactinomycin, Cyclophosphamide |
| VACAdr | Vincristine, Cyclophosphamide, Doxorubicin, Dactinomycin, |
| | Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, Prednisone |
| VC | Vinorelbine, Cisplatin |
| VCAP | Vincristine, Cyclophosphamide, Doxorubicin, Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or Mitoxantrone |
| “8 in 1” | Methylprednisolone, Vincristine, Lomustine, Procarbazine, |
| | Hydroxyurea, Cisplatin, Cytarabine, Dacarbazine |

In addition to conventional anti-cancer agents, the agent of the subject method can also be compounds and antisense RNA, RNAi or other polynucleotides to inhibit the expression of the cellular components that contribute to unwanted cellular proliferation that are targets of conventional chemotherapy. Such targets are, merely to illustrate, growth factors, growth factor receptors, cell cycle regulatory proteins, transcription factors, or signal transduction kinases.

The method of present invention is advantageous over combination therapies known in the art because it allows conventional anti-cancer agent to exert greater effect at lower dosage. In preferred embodiment of the present invention, the effective dose (ED50) for a anti-cancer agent or combination of conventional anti-cancer agents when used in combination with a Na⁺/K⁺-ATPase inhibitor (e.g. cardiac glycoside) is at least 5 fold less than the ED₅₀ for the anti-cancer agent alone. Conversely, the therapeutic index (TI) for such anti-cancer agent or combination of such anti-cancer agent when used in combination with a Na⁺/K⁺-ATPase inhibitor (e.g. cardiac glycoside) is at least 5 fold greater than the TI for conventional anti-cancer agent regimen alone.

C. Other Treatment Methods

In yet other embodiments, the subject method combines a Na⁺/K⁺-ATPase inhibitor (e.g. cardiac glycoside) with radiation therapies, including ionizing radiation, gamma radiation, or particle beams.

D. Administration

The Na⁺/K⁺-ATPase inhibitor (e.g. cardiac glycoside), or a combination containing a Na⁺/K⁺-ATPase inhibitor (e.g. cardiac glycoside) may be administered orally, parenterally by intravenous injection, transdermally, by pulmonary inhalation, by intravaginal or intrarectal insertion, by subcutaneous implantation, intramuscular injection or by injection directly into an affected tissue, as for example by injection into a tumor site. In some instances the materials may be applied topically at the time surgery is carried out. In another instance the topical administration may be ophthalmic, with direct application of the therapeutic composition to the eye.

In a preferred embodiment, the subject Na⁺/K⁺-ATPase inhibitors (e.g. cardiac glycosides) are administered to a patient by using osmotic pumps, such as Alzet® Model 2002 osmotic pump. Osmotic pumps provides continuous delivery of test agents, thereby eliminating the need for frequent, round-the-clock injections. With sizes small enough even for use in mice or young rats, these implantable pumps have proven invaluable in predictably sustaining compounds at therapeutic levels, avoiding potentially toxic or misleading side effects.

To meet different therapeutic needs, ALZET's osmotic pumps are available in a variety of sizes, pumping rates, and durations. At present, at least ten different pump models are available in three sizes (corresponding to reservoir volumes of 100 μL, 200 μL and 2 mL) with delivery rates between 0.25 μL/hr and 10 μL/hr and durations between one day to four weeks.

While the pumping rate of each commercial model is fixed at manufacture, the dose of agent delivered can be adjusted by varying the concentration of agent with which each pump is filled. Provided that the animal is of sufficient size, multiple pumps may be implanted simultaneously to achieve higher delivery rates than are attainable with a single pump. For more prolonged delivery, pumps may be serially implanted with no ill effects. Alternatively, larger pumps for larger patients, including human and other non-human mammals may be custom manufactured by scaling up the smaller models.

The materials are formulated to suit the desired route of administration. The formulation may comprise suitable excipients include pharmaceutically acceptable buffers, stabilizers, local anesthetics, and the like that are well known in the art. For parenteral administration, an exemplary formulation may be a sterile solution or suspension; For oral dosage, a syrup, tablet or palatable solution; for topical application, a lotion, cream, spray or ointment; for administration by inhalation, a microcrystalline powder or a solution suitable for nebulization; for intravaginal or intrarectal administration, pessaries, suppositories, creams or foams. Preferably, the route of administration is parenteral, more preferably intravenous.

Exemplification

The following examples are for illustrative purpose only, and should in no way be construed to be limiting in any respect of the claimed invention.

The exemplary cardiac glycosides used in following studies are referred to as BNC-1 and BNC-4.

BNC-1 is ouabain or g-Strophanthin (STRODIVAL®), which has been used for treating myocardial infarction. It is a colorless crystal with predicted IC₅₀ of about 0.009-0.035 μg/mL and max. plasma concentration of about 0.03 μg/mL. According to the literature, its plasma half-life in human is about 20 hours, with a range of between 5-50 hours. Its common formulation is injectable. The typical dose for current indication (i.v.) is about 0.25 mg, up to 0.5 mg /day.

BNC-4 is proscillaridin (TALUSIN®), which has been approved for treating chronic cardiac insufficiency in Europe. It is a colorless crystal with predicted IC₅₀ of about 0.002-0.008 μg/mL and max. plasma concentration of about 0.1 μg/mL. According to the literature, its plasma half-life in human is about 40 hours. Its common available formulation is a tablet of 0.25 or 0.5 mg. The typical dose for current indication (p.o.) is about 1.5 mg /day.

### EXAMPLE I

### Sentinel Line Plasmid Construction and Virus Preparation

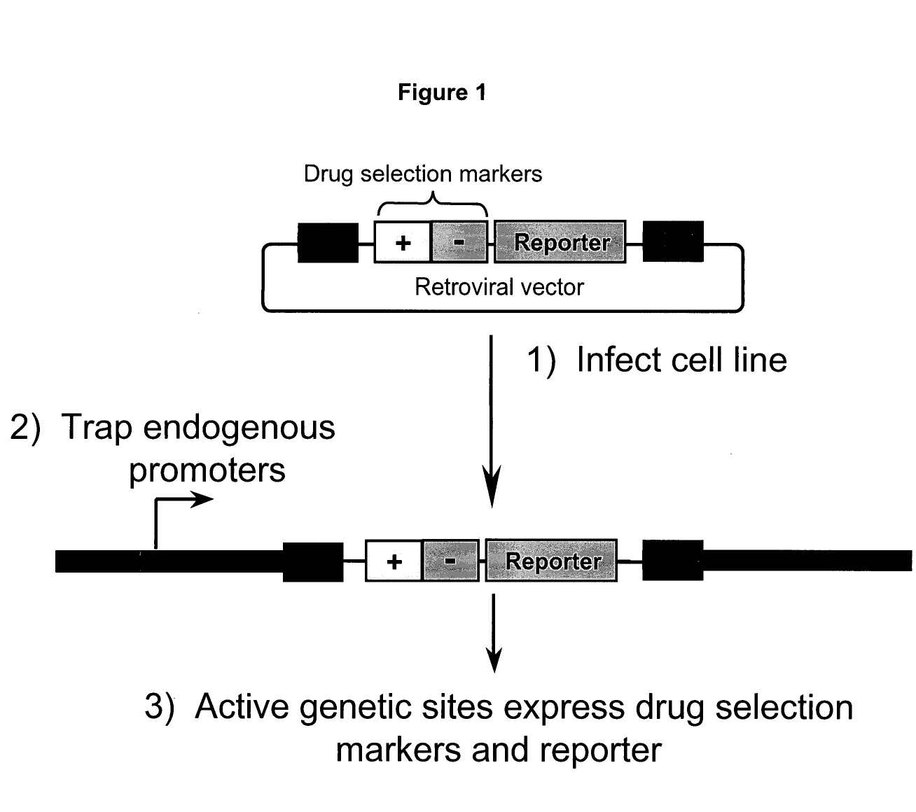

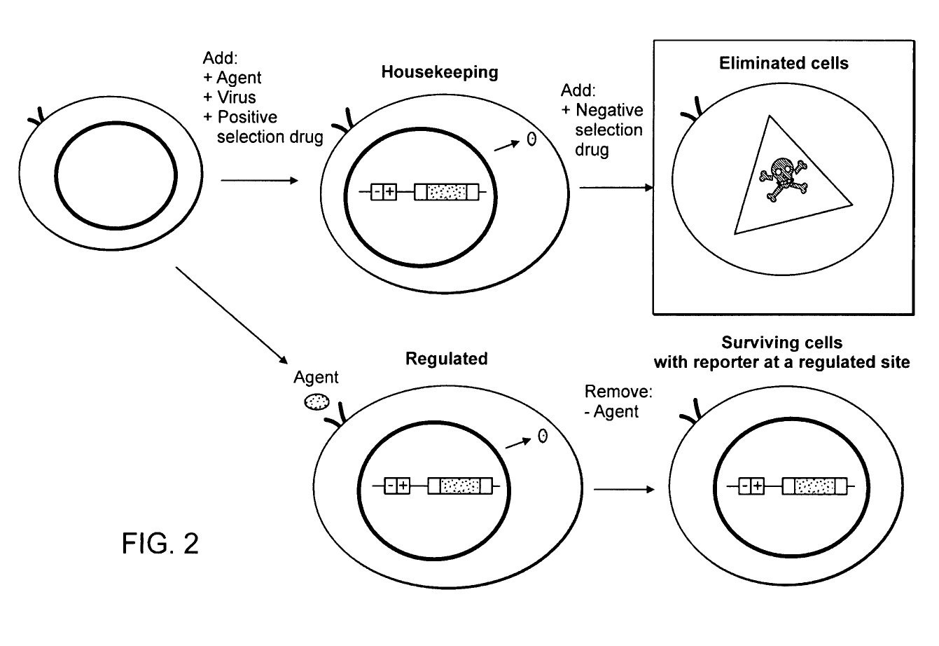

FIG. 1 is a schematic drawing of the Sentinel Line promoter trap system, and its use in identifying regulated genetic sites and in reporting pathway activity. Briefly, the promoter-less selection markers (either positive or negative selection markers, or both) and reporter genes (such as beta-gal) are put in a retroviral vector (or other suitable vectors), which can be used to infect target cells. The randomly inserted retroviral vectors may be so positioned that an active upstream heterologous promoter may initiate the transcription and translation of the selectable markers and reporter gene(s). The expression of such selectable markers and/or reporter genes is indicative of active genetic sites in the particular host cell.

In one exemplary embodiment, the promoter trap vector BV7 was derived from retrovirus vector pQCXIX (BD Biosciences Clontech) by replacing sequence in between packaging signal (Psi⁺) and 3′ LTR with a cassette in an opposite orientation, which contains a splice acceptor sequence derived from mouse engrailed 2 gene (SA/en2), an internal ribosomal entry site (IRES), a LacZ gene, a second IRES, and fusion gene TK:Sh encoding herpes virus thymidine kinase (HSV-tk) and phleomycin followed by a SV40 polyadenylation site. BV7 was constructed by a three-way ligation of three equal molar DNA fragments. Fragment 1 was a 5 kb vector backbone derived from pQCXIX by cutting plasmid DNA extracted from a Dam-bacterial strain with Xho I and Cla I (Dam-bacterial strain was needed here because Cla I is blocked by overlapping Dam methylation). Fragment 2 was a 2.5 kb fragment containing an IRES and a TK:Sh fusion gene derived from plasmid pIREStksh by cutting Dam-plasmid DNA with Cla I and Mlu I. pIREStksh was constructed by cloning TK:Sh fragment from pMODtksh (InvivoGen) into pIRES (BD Biosciences Clontech). Fragment 3 was a 5.8 kb SA/en2-IRES-LacZ fragment derived from plasmid pBSen2IRESLacZ by cutting with BssH II (compatible end to Mlu I) and Xho I. pBSen2IRESLacZ was constructed by cloning IRES fragment from pIRES and LacZ fragment from pMODLacZ (InvivoGen) into plasmid pBSen2.

To prepare virus, packaging cell line 293T was co-transfected with three plasmids BV7, pVSV-G (BD Biosciences Clontech) and pGag-Pol (BD Biosciences Clontech) in equal molar concentrations by using Lipofectamine 2000 (InvitroGen) according to manufacturer's protocol. First virus “soup” (supernatant) was collected 48 hours after transfection, second virus “soup” was collected 24 hours later. Virus particles were pelleted by centrifuging at 25,000 rpm for 2 hours at 4° C. Virus pellets were re-dissolved into DMEM/10% FBS by shaking overnight. Concentrated virus solution was aliquot and used freshly or frozen at −80° C.

### EXAMPLE II

### Sentinel Line Generation

Target cells were plated in 150 mm tissue culture dishes at a density of about 1×10⁶/plate. The following morning cells were infected with 250 μl of Bionaut Virus #7 (BV7) as prepared in Example I, and after 48 hr incubation, 20 μg/ml of phleomycin was added. 4 days later, media was changed to a reduced serum (2%FBS) DMEM to allow the cells to rest. 48 h later, ganciclovir (GCV) (0.4μM, sigma) was added for 4 days (media was refreshed on day 2). One more round of phleomycin selection followed (20 μg/ml phleomycin for 3 days). Upon completion, media was changed to 20%FBS DMEM to facilitate the outgrowths of the clones. 10 days later, clones were picked and expanded for further analysis and screening.

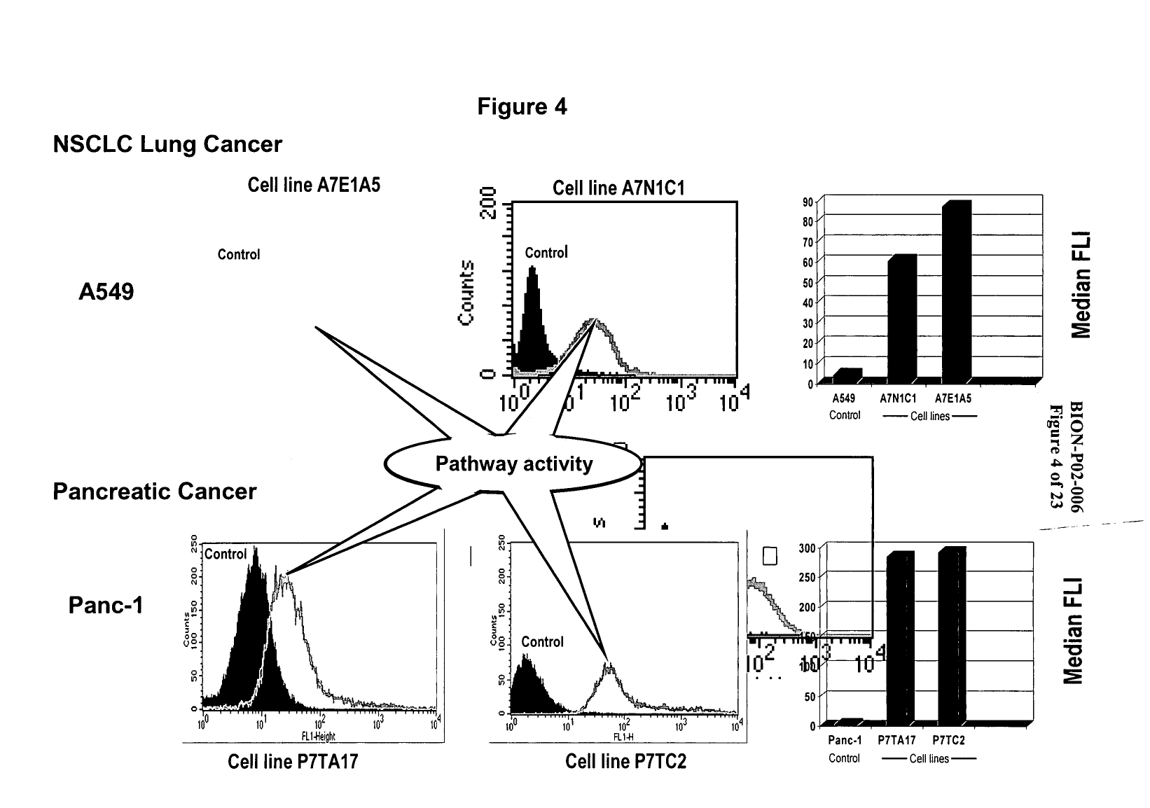

Using this method, several Sentinel Lines were generated to report activity of genetic sites activated by hypoxia pathways (FIG. 4). These Sentinel lines were generated by transfecting A549 (NSCLC lung cancer) and Panc-1 (pancreatic cancer) cell lines with the subject gene-trap vectors containing E. coli LacZ-encoded β-galactosidase (β-gal) as the reporter gene (FIG. 4). The β-gal activity in Sentinel Lines (green) was measured by flow cytometry using a fluorogenic substrate fluoresescein di-beta-D-galactopyranoside (FDG). The autofluorescence of untransfected control cells is shown in purple. The graphs indicate frequency of cells (y-axis) and intensity of fluorescence (x-axis) in log scale. The bar charts on the right depict median fluorescent units of the FACS curves. They indicate a high level of reporter activity at the targeted site.

### EXAMPLE III

### Cell Culture and Hypoxic Conditions

All cell lines can be purchased from ATCC, or obtained from other sources.

A549 (CCL-185) and Panc-1 (CRL-1469) were cultured in Dulbecco's Modified Eagle's Medium (DMEM), Caki-1 (HTB-46) in McCoy's Sa modified medium, Hep3B (HB-8064) in MEM-Eagle medium in humidified atmosphere containing 5% CO₂ at 37° C. Media was supplemented with 10% FBS (Hyclone; SH30070.03), 100 μg/ml penicillin and 50 μg/ml streptomycin (Hyclone).

To induce hypoxia conditions, cells were placed in a Billups-Rothenberg modular incubator chamber and flushed with artificial atmosphere gas mixture (5% CO₂, 1% O₂, and balance N₂). The hypoxia chamber was then placed in a 37° C. incubator. L-mimosine (Sigma, M-0253) was used to induce hypoxia-like HIF-1-alpha expression. Proteosome inhibitor, MG132 (Calbiochem, 474791), was used to protect the degradation of HIF-1-alpha. Cycloheximide (Sigma, 4859) was used to inhibit new protein synthesis of HIF-1-alpha. Catalase (Sigma, C3515) was used to inhibit reactive oxygen species (ROS) production.

### EXAMPLE IV

### Identification of Trapped Genes

Once a Sentinel Line with a desired characteristics was established, it might be helpful to determine the active promoter under which control the markers/reporter genes are expressed. To do so, total RNAs were extracted from cultured Sentinel Line cells by using, for example, RNA-Bee RNA Isolation Reagent (TEL-TEST, Inc.) according to the manufacturer's instructions. Five prime ends of the genes that were disrupted by the trap vector BV7 were amplified by using BD SMART RACE cDNA Amplification Kit (BD Biosciences Clontech) according to the manufacturer's protocol. Briefly, 1 μg total RNA prepared above was reverse-transcribed and extended by using BD PowerScriptase with 5′ CDS primer and BD SMART II Oligo both provided by the kit. PCR amplification were carried out by using BD Advantage 2 Polymerase Mix with Universal Primer A Mix provided by the kit and BV7 specific primer 5′Rsa/ires (gacgcggatcttccgggtaccgagctcc, 28 mer). 5′Rsa/ires located in the junction of SA/en2 and IRES with the first 7 nucleotides matching the last 7 nucleotides of SA/en2 in complementary strand. 5′ RACE products were cloned into the TA cloning vector pCR2.1 (InvitroGen) and sequenced. The sequences of the RACE products were analyzed by using the BLAST program to search for homologous sequences in the database of GenBank. Only those hits which contained the transcript part of SA/en2 were considered as trapped genes.

Using this method, the upstream promoters of several Sentinel Lines generated in Example II were identified (see below). The identity of these trapped genes validate the clinical relevance of these Sentinel Lines™, and can be used as biomarkers and surrogate endpoints in clinical trials.

| Sentinel Lines | Genetic Sites | Gene Profile |
|---|---|---|
| A7N1C1 | Essential Antioxidant | Tumor cell-specific gene, over |
| | | expressed in lung tumor cells |
| A7N1C6 | Chr. 3, BAC, map to 3p | novel |
| A7I1C1 | Pyruvate Kinase | Described biomarker for |
| | (PKM 2), Chr. 15 | NSCLC |
| A6E2A4 | 6q14.2-16.1 | Potent angiogenic activity |
| A7I1D1 | Chr. 7, BAC | novel |

### EXAMPLE V

### Western Blots

For HIF-1-alpha Western blots, Hep3B cells were seeded in growth medium at a density of 7×10⁶ cells per 100 mm dish. Following 24-hour incubation, cells were subjected to hypoxic conditions for 4 hours to induce HIF-1-alpha expression together with an agent such as 1 μM BNC-1. The cells were harvested and lysed using the Mammalian Cell Lysis kit (Sigma, M-0253). The lysates were centrifuged to clear insoluble debris, and total protein contents were analyzed with BCA protein assay kit (Pierce, 23225). Samples were fractionated on 3-8% Tris-Acetate gel (Invitrogen NUPAGE system) by sodium dodecyl sulfate (SDS)-polyacrylamide gel electropherosis and transferred onto nitrocellulose membrane. HIF-1-alpha protein was detected with anti-HIF-1-alpha monoclonal antibody (BD Transduction Lab, 610959) at a 1:500 dilution with an overnight incubation at 4° C. in Tris-buffered solution-0.1% Tween 20 (TBST) containing 5% dry non-fat milk. Anti-Beta-actin monoclonal antibody (Abcam, ab6276-100) was used at a 1:5000 dilution with a 30-minute incubation at room temperature. Immunoreactive proteins were detected with stabilized goat-anti mouse HRP conjugated antibody (Pierce, 1858413) at a 1:10,000 dilution. The signal was developed using the West Femto substrate (Pierce, 34095).

We examined the inhibitory effect of BNC-1 on HIF-1 alpha synthesis. 24 hours prior to treatment, Hep3B cells were seeded in growth medium. To show that BNC-1 inhibits HIF-1-alpha expression in a concentration dependent manner, cells were treated with 1 μM BNC-1 together with the indicated amount of MG132 under hypoxic conditions for 4 hours. To understand specifically the impact of BNC-1 on HIF-1 alpha synthesis, Hep3B cells were treated with MG132 and 1 μM BNC under normoxic conditions for the indicated time points. The observed expression is accounted by protein synthesis.

We examined the role of BNC-1 on the degradation rate of HIF-1 alpha. 24 hours prior to treatment, Hep3B cells were seeded in growth medium. The cells were placed in hypoxic conditions for 4 hours for HIF-1-alpha accumulation. The protein synthesis inhibitor, cycloheximide (100 μM) together with 1 μM BNC-1 were added to the cells and kept in hypoxic conditions for the indicate time points.

To induce HIF-1-alpha expression using an iron chelator, L-mimosine was added to Hep3B cells, seeded 24 hours prior, and placed under normoxic conditions for 24 hours.

### EXAMPLE VI

### Sentinel Line Reporter Assays

The expression level of beta-galactosidase gene in sentinel lines was determined by using a fluorescent substrate fluorescein di-B-D-Galactopyranside (FDG, Marker Gene Tech, #M0250) introduced into cells by hypotonic shock. Cleavage by beta-galactosidase results in the production of free fluorescein, which is unable to cross the plasma membrane and is trapped inside the beta-gal positive cells. Briefly, the cells to be analyzed are trypsinized, and resuspended in PBS containing 2 mM FDG (diluted from a 10 mM stock prepared in 8:1:1 mixture of water: ethanol: DMSO). The cells were then shocked for 4 minutes at 37° C. and transferred to FACS tubes containing cold 1×PBS on ice. Samples were kept on ice for 30 minutes and analyzed by FACS in FLI channel.

### EXAMPLE VII

### Testing Standard Chemotherapeutic Agents

Sentinel Line cells with beta-galactosidase reporter gene were plated at 1×10⁵ cells/10 cm dish. After overnight incubation, the cells were treated with standard chemotherapeutic agents, such as mitoxantrone (8 nM), paclitaxel (1.5 nM), carboplatin (15 EM), gemcitabine (2.5 nM), in combination with one or more BNC compounds, such as BNC-1 (10 nM), BNC2 (2 μM), BNC3 (100 μM) and BNC-4 (10 nM), or a targeted drug, Iressa (4 EM). After 40 hrs, the cells were trypsinized and the expression level of reporter gene was determined by FDG loading.

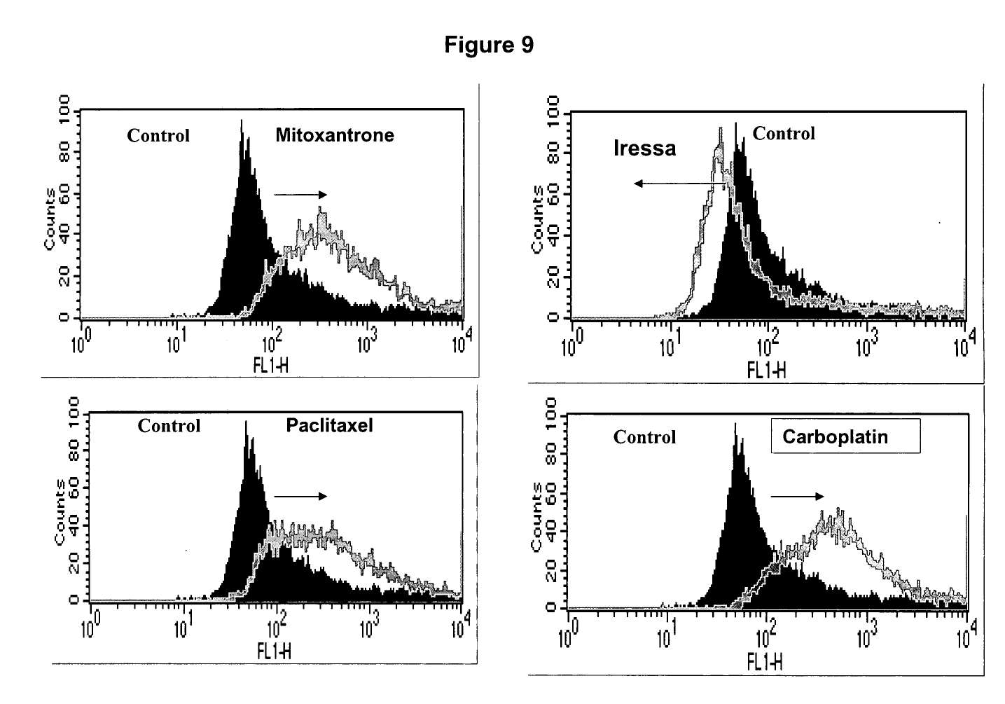

When tested in the Sentinel Lines, mitoxanthrone, paclitaxel, and carboplatin each showed increases in cell death and reporter activity (see FIG. 9). No effect had been expected from the cytotoxic agents because of their nonspecific mechanisms of action (MOA), making their increased reporter activity in HIF-sensitive cell lines surprising. These results provide a previously unexplored link between the development of chemotherapy resistance and induction of the hypoxia response in cells treated with anti-neoplastic agents. Iressa, on the other hand, a known blocker of EGFR-mediated HIF-1 induction, showed a reduction in reporter activity when tested. The Sentinel Lines thus provide a means to differentiate between a cytotoxic agent and a targeted drug.

### EXAMPLE VIII

### Pharmacokinetic (PK) Analysis

The following protocol can be used to conduct pharmacokinetic analysis of any compounds of the invention. To illustrate, BNC-1 is used as an example.

Nude mice were dosed i.p. with 1, 2, or 4 mg/kg of BNC-1. Venous blood samples were collected by cardiac puncture at the following 8 time points: 5 min, 15 min, 30 min, 45 min, 1 hr, 2 hr, 4 hr, 8 hr, and 24 hr. For continuous BNC-1 treatment, osmotic pumps (such as Alzet® Model 2002) were implanted s.c. between the shoulder blades of each mouse. Blood was collected at 24 hr, 48 hr and 72 hr. Triplicate samples per dose (i.e. three mice per time point per dose) were collected for this experiment.

Approximately 0.100 mL of plasma was collected from each mouse using lithium heparin as anticoagulant. The blood samples were processed for plasma as individual samples (no pooling). The samples were frozen at −70° C. (±10° C.) and transferred on dry ice for analysis by HPLC.

For PK analysis plasma concentrations for each compound at each dose were analyzed by non-compartmental analysis using the software program WinNonlin®. The area under the concentration vs time curve AUC (0-Tf) from time zero to the time of the final quantifiable sample (Tf) was calculated using the linear trapezoid method. AUC is the area under the plasma drug concentration-time curve and is used for the calculation of other PK parameters. The AUC was extrapolated to infinity (0-Inf) by dividing the last measured concentration by the terminal rate constant (k), which was calculated as the slope of the log-linear terminal portion of the plasma concentrations curve using linear regression. The terminal phase half-life (t_{1/2}) was calculated as 0.693/k and systemic clearance (Cl) was calculated as the dose(mg/kg)/AUC(Inf). The volume of distribution at steady-state (Vss) was calculated from the formula:
V_{SS}=dose(AUMC)/(AUC)²

where AUMC is the area under the first moment curve (concentration multiplied by time versus time plot) and AUC is the area under the concentration versus time curve. The observed maximum plasma concentration (Cₘₐₓ) was obtained by inspection of the concentration curve, and Tₘₐₓ is the time at when the maximum concentration occurred.

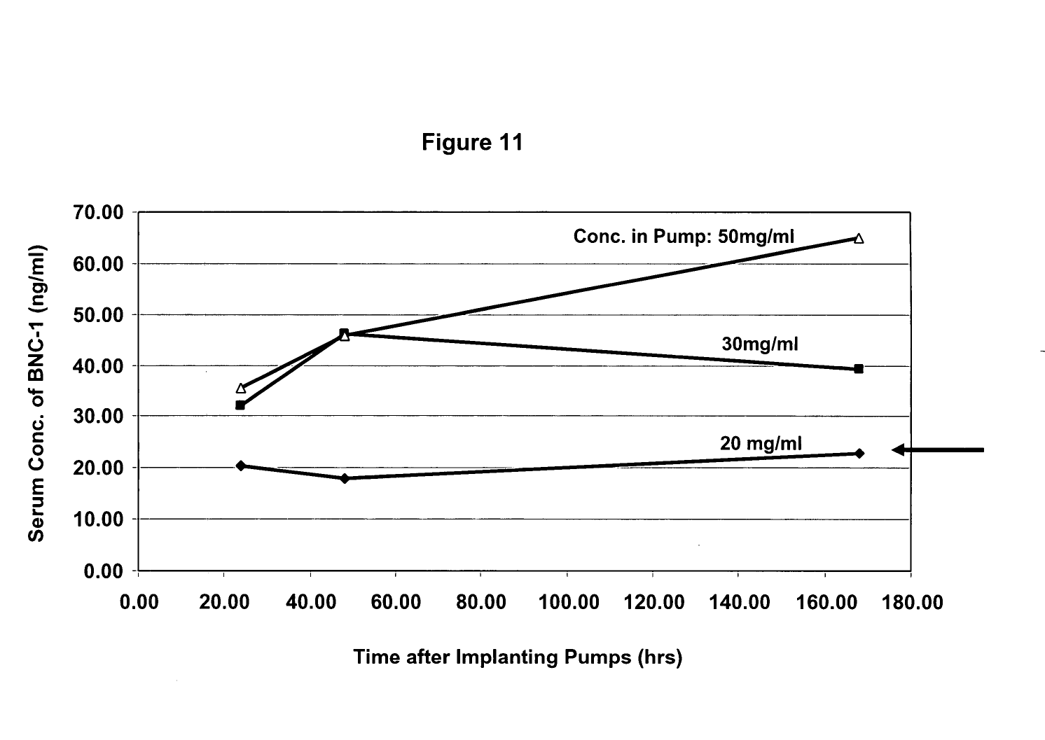

FIG. 11 shows the result of a representative pharmacokinetic analysis of BNC-1 delivered by osmotic pumps. Osmotic pumps (Model 2002, Alzet Inc) containing 200 μl of BNC-1 at 50, 30 or 20 mg/ml in 50% DMSO were implanted subcutaneously into nude mice. Mice were sacrificed after 24, 48 or 168 hrs, and plasma was extracted and analyzed for BNC-1 by LC-MS. The values shown are average of 3 animals per point.

### EXAMPLE IX

### Human Tumor Xenograft Models

Female nude mice (nu/nu) between 5 and 6 weeks of age weighing approximately 20 g were implanted subcutaneously (s.c.) by trocar with fragments of human tumors harvested from s.c. grown tumors in nude mice hosts. When the tumors were approximately 60-75 mg in size (about 10-15 days following inoculation), the animals were pair-matched into treatment and control groups. Each group contains 8-10 mice, each of which was ear tagged and followed throughout the experiment.

The administration of drugs or controls began the day the animals were pair-matched (Day 1). Pumps (Alzet® Model 2002) with a flow rate of 0.5 μl/hr were implanted s.c. between the shoulder blades of each mice. Mice were weighed and tumor measurements were obtained using calipers twice weekly, starting Day 1. These tumor measurements were converted to mg tumor weight by standard formula, (W²×L)/2. The experiment is terminated when the control group tumor size reached an average of about 1 gram. Upon termination, the mice were weighed, sacrificed and their tumors excised. The tumors were weighed and the mean tumor weight per group was calculated. The change in mean treated tumor weight/the change in mean control tumor weight×100 (dT/dC) is subtracted from 100% to give the tumor growth inhibition (TGI) for each group.

### EXAMPLE X

### Cardiac Glycoside Compounds Inhibits HIF-1α Expression

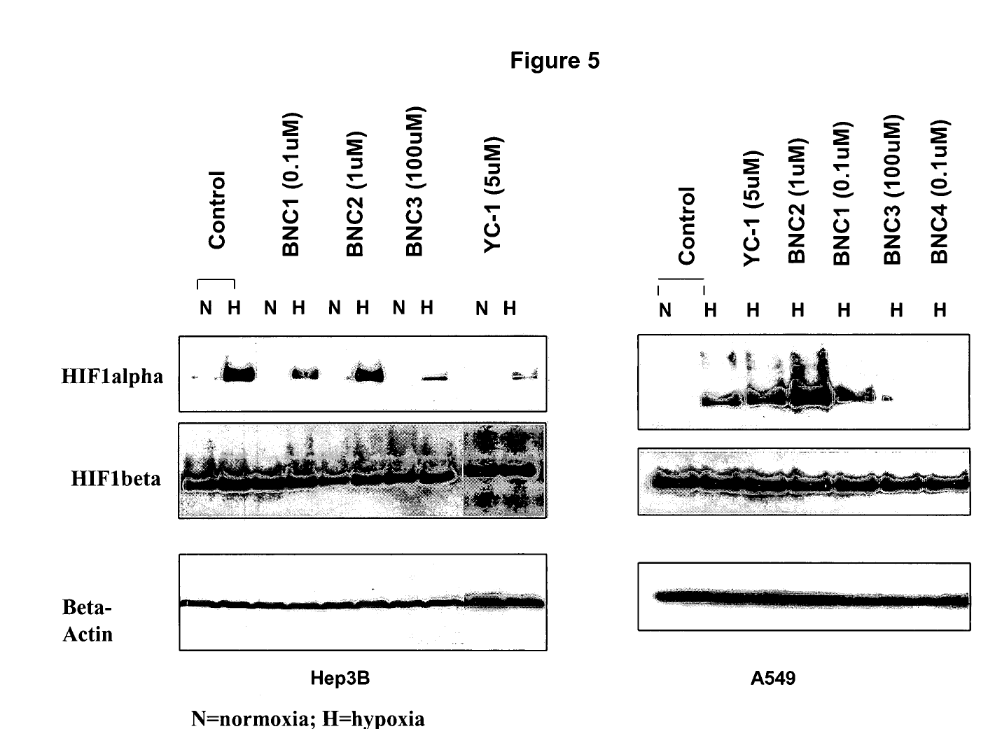

Cardiac glycoside compounds of the invention targets and inhibits the expression of HIF 1α based on Western Blot analysis using antibodies specific for HIF-1α (FIG. 5).

Hep3B or A549 cells were cultured in complete growth medium for 24 hours and treated for 4 hrs with the indicated cardiac glycoside compounds or controls under normoxia (N) or hypoxia (H) conditions. The cells were lysed and proteins were resolved by SDS-PAGE and transferred to a nylon membrane. The membrane was immunoblotted with anti-HIF-1α and anti-HIF-1β MAb, and anti-beta-actin antibodies.

In Hep3B cells, various effective concentrations of BNC compounds (cardiac glycoside compounds of the invention) inhibits the expression of HIF-1α, but not HIF-1β. The basic observation is the same, with the exception of BNC2 at 1 μM concentration.

To study the mechanism of HIF-1α inhibition by the subject cardiac glycoside compounds, Hep3B cells were exposed to normoxia or hypoxia for 4 hrs in the presence or absence of: an antioxidant enzyme and reactive oxygen species (ROS) scavenger catalase (1000 U), prolyl-hydroxylase (PHD) inhibitor L-mimosine, or proteasome inhibitor MG132 as indicated. HIF-1α and β-actin protein level was determined by western blotting.

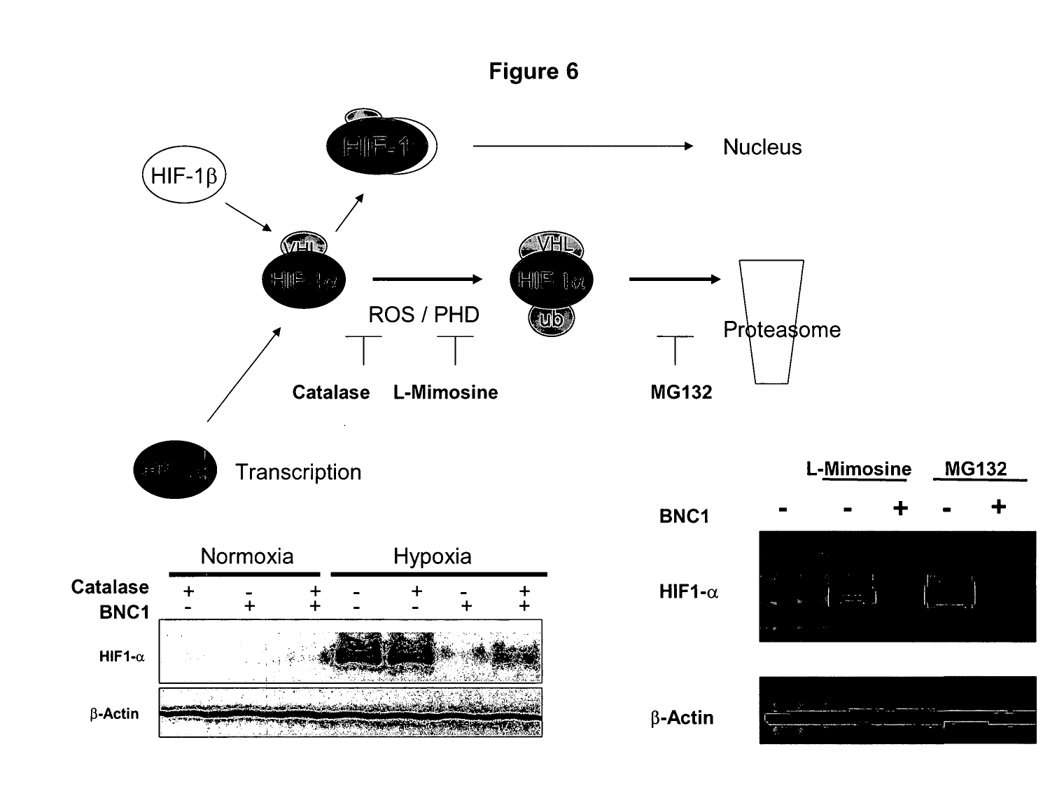

FIG. 6 indicates that the cardiac glycoside compound BNC-1 may inhibits steady state HIF-1α level through inhibiting the synthesis of HIF-1α.

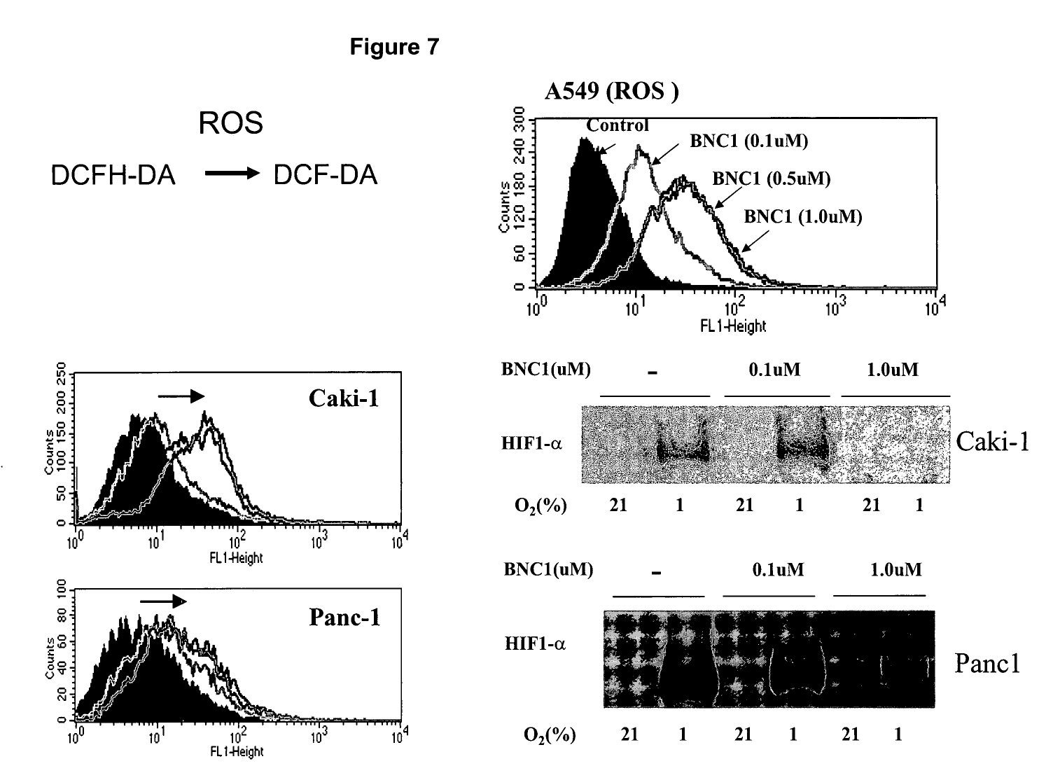

In a related study, tumor cell line A549(ROS) were incubated in normoxia in the absence (control) or presence of different amounts of BNC-1 for 4 hrs. Thirty minutes prior to the termination of incubation period, 2,7-dichlorofluorescin diacetate (CFH-DA, 10 mM) was added to the cells and incubated for the last 30 min at 37° C. The ROS levels were determined by FACS analysis. HIF-1α protein accumulation in Caki-1 and Panc-1 cells was determined by western blotting after incubating the cells for 4 hrs in normoxia (21% O₂) or hypoxia (1% O₂) in the presence or absence of BNC-1. FIG. 7 indicates that BNC-1 induces ROS production (at least as evidenced by the A549(ROS) Sentinel Lines), and inhibits HIF-1α protein accumulation in the test cells.

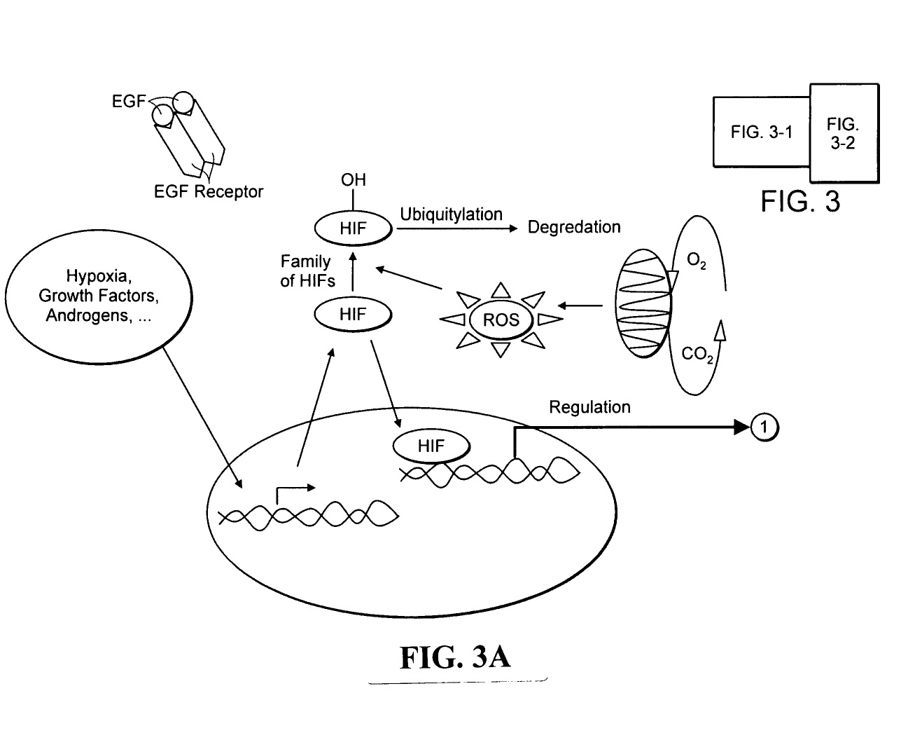

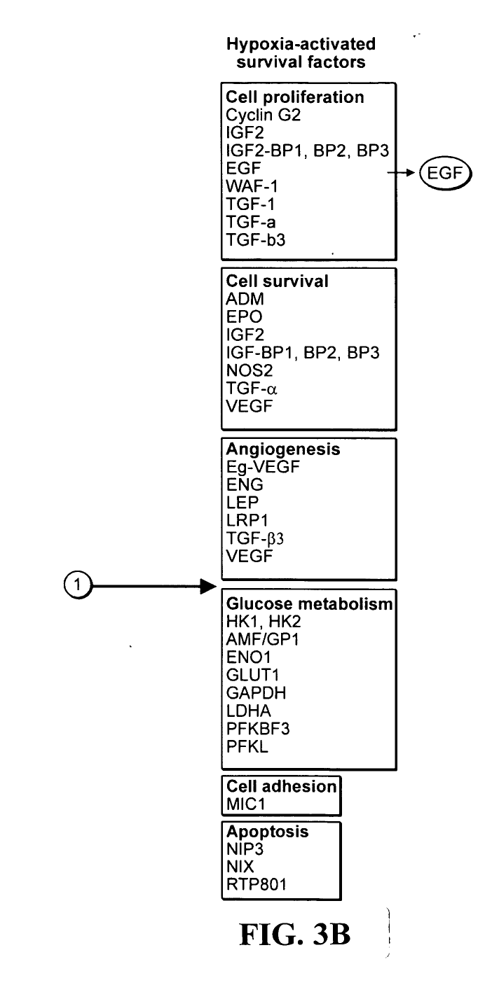

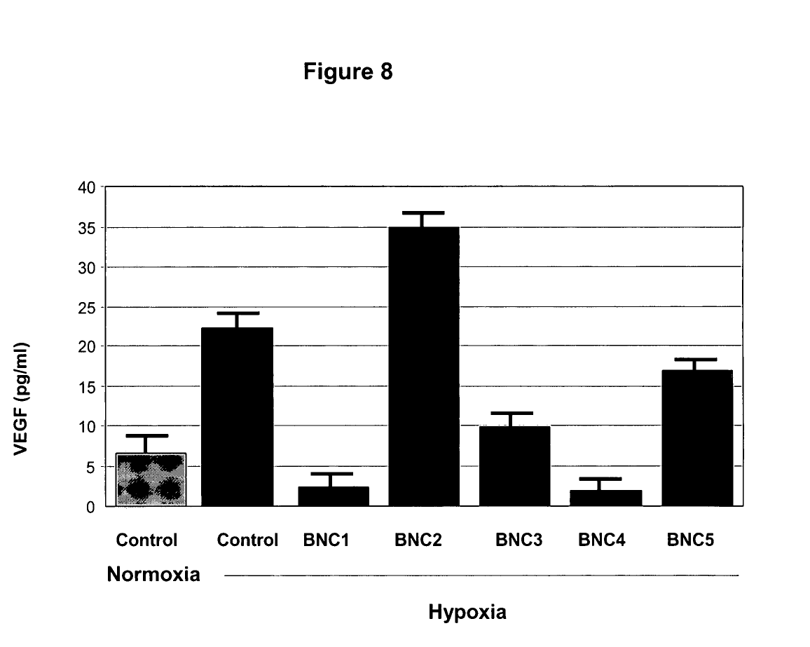

FIG. 8 also demonstrates that the cardiac glycoside compounds BNC-1 and BNC-4 directly or indirectly inhibits in tumor cells the secretion of the angiogenesis factor VEGF, which is a downstream effector of HIF-1α (see FIG. 3). In contrast, other non-cardiac glycoside compounds, BNC2, BNC3 and BNC5, do not inhibit, and in fact greatly enhances VEGF secretion.

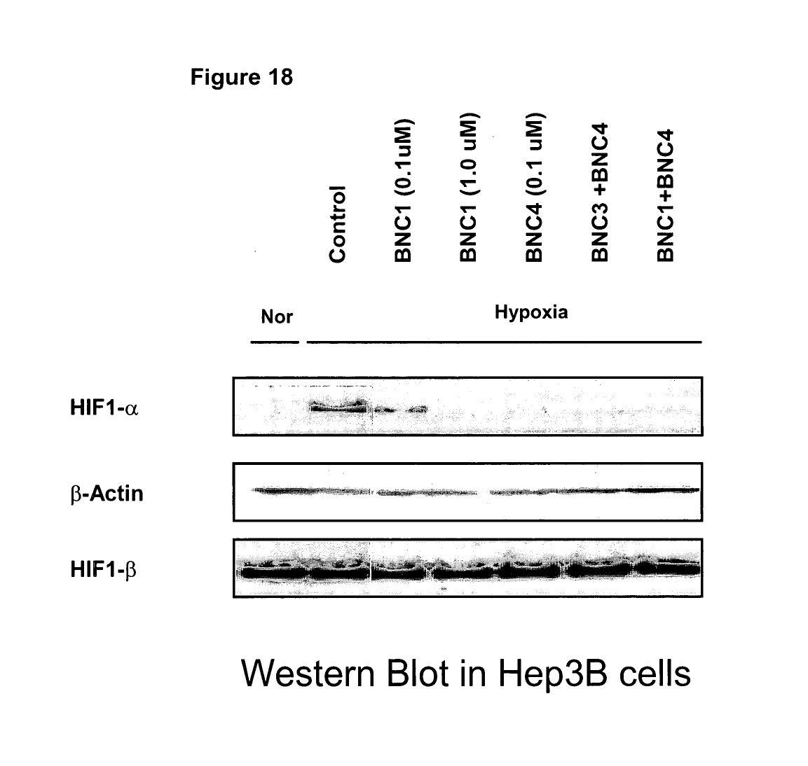

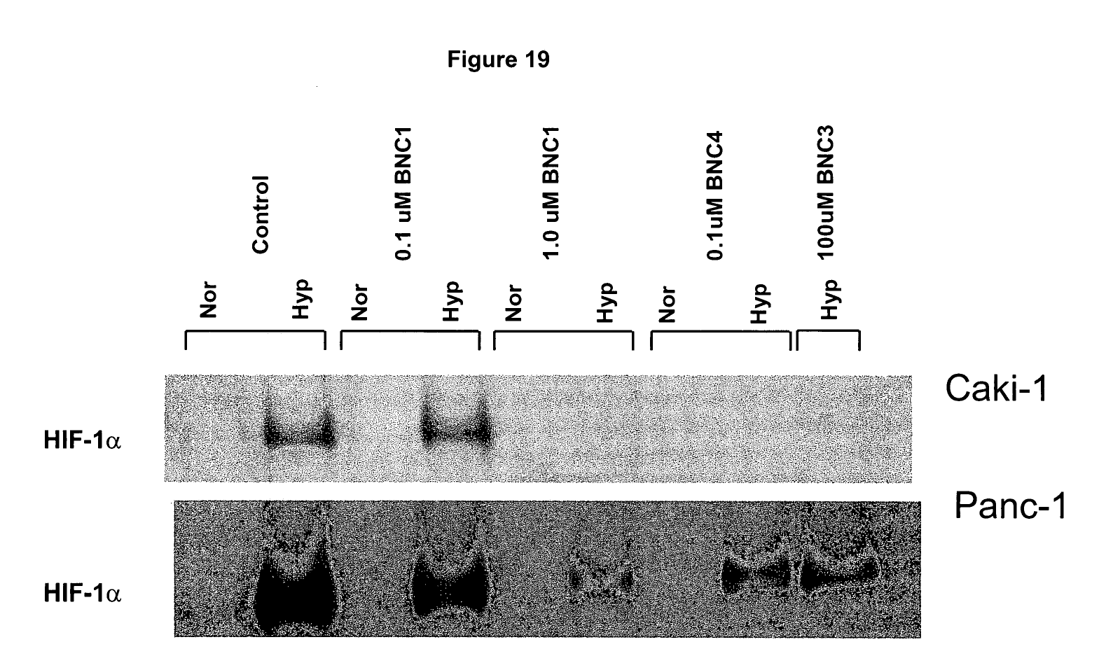

FIGS. 18 and 19 compared the ability of BNC-1 and BNC-4 in inhibiting hypoxia-mediated HIF-1α induction in human tumor cells. The figures show result of immunoblotting for HIF-1α, HIF-1β and β-actin (control) expression, in Hep3B, Caki-1 or Panc-1 cells treated with BNC-1 or BNC-4 under hypoxia. The results indicate that BNC-4 is even more potent (about 10-times more potent) than BNC-1 in inhibiting HIF-1α expression.

### EXAMPLE XI

### Neutralization of Gemcitabine-Induced Stress Response as Measured in A549 Sentinel Line

The cardiac glycoside compounds of the invention were found to be able to neutralize Gemcitabine-induced stress response in tumor cells, as measured in A549 Sentinal Lines.

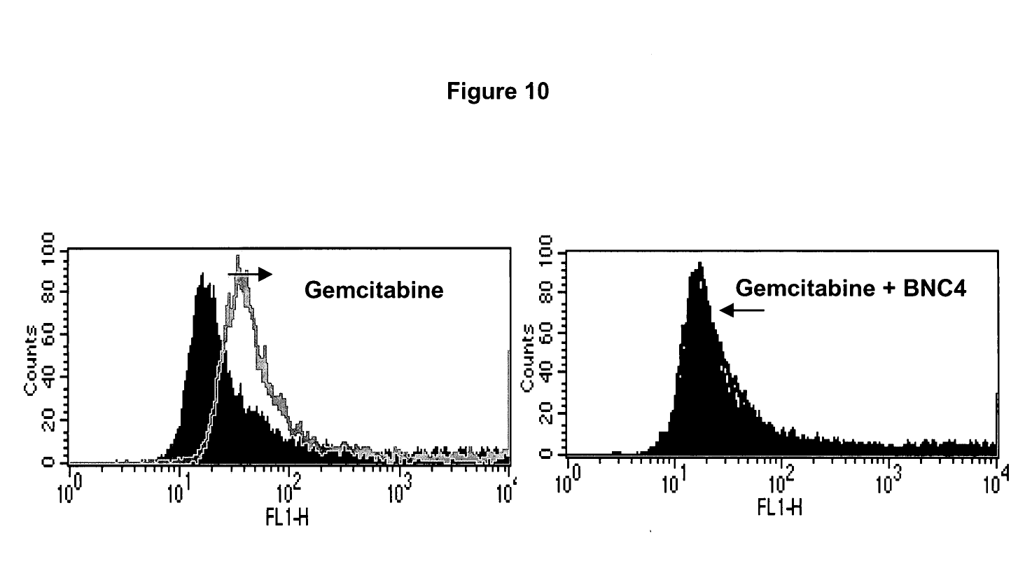

In experiments of FIG. 10, the A549 sentinel line was incubated with Gemcitabine in the presence or absence of indicated Bionaut compounds (including the cardiac glycoside compound BNC-4) for 40 hrs. The reporter activity was measured by FACS analysis.

It is evident that at least BNC-4 can significantly shift the reporter activity to the left, such that Gemcitabine and BNC-4-treated cells had the same reporter activity as that of the control cells. In contrast, cells treated with only Gemcitabine showed elevated reporter activity.

### EXAMPLE XII

### Effect of BNC-1 Alone or in Combination with Standard Chemotherapy on Growth of Xenografted Human Pancreatic Tumors in Nude Mice

To test the ability of BNC-1 to inhibit xenographic tumor growth in nude mice, either along or in combination with a standard chemotherapeutic agent, such as Gemcitabine, Panc-1 tumors were injected subcutaneously (sc) into the flanks of male nude mice. After the tumors reached 80 mg in size, osmotic pumps (model 2002, Alzet Inc., flow rate 0.5 μl/hr) containing 20 mg/ml of BNC-1 were implanted sc on the opposite sides of the mice. The control animals received pumps containing vehicle (50% DMSO in DMEM). The mice treated with standard chemotherapy agent received intra-peritoneal injections of Gemcitabine at 40 mg/kg every 3 days for 4 treatments (q3d×4). Each data point represent average tumor weight (n=8) and error bars indicate SEM.

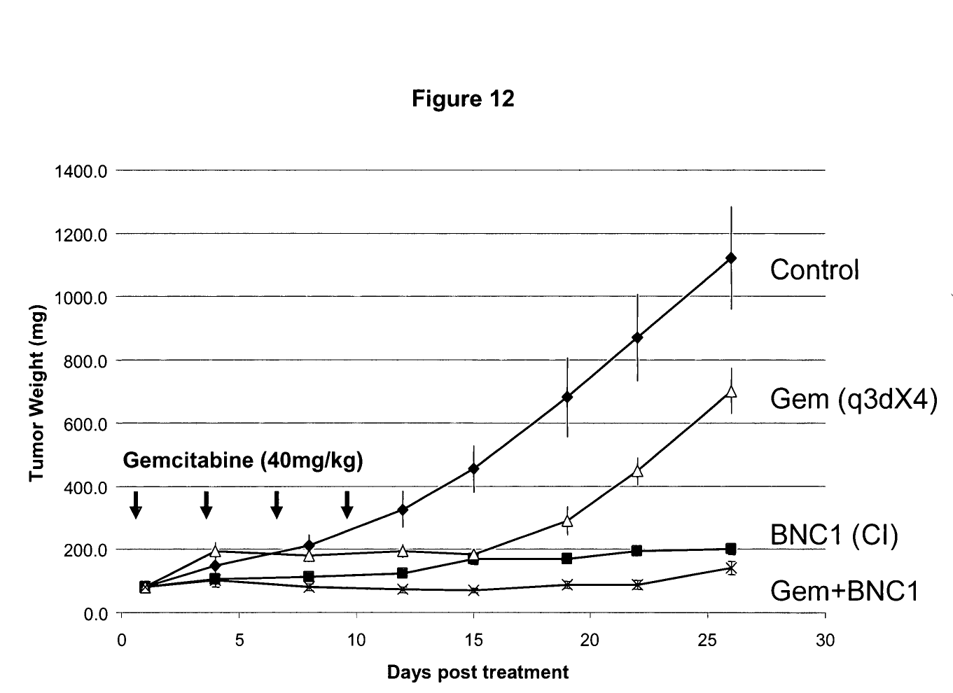

FIG. 12 indicates that, at the dosage tested, BNC-1 alone can significantly reduce tumor growth in this model. This anti-tumor activity is additive when BNC-1 is co-administered with a standard chemotherapeutic agent Gemcitabine. Results of the experiment is listed below:

| | | Final Tumor | | |
|---|---|---|---|---|
| Group | | weight | | |
| (Animal No.) | Dose/Route | Day 25 (Mean) | SEM | % TGI |
| Control (8) | Vehicle/i.v. | 1120.2 | 161.7 | — |
| BNC-1 (8) | 20 mg/ml; s.c.; C.I. | 200 | 17.9 | 82.15 |
| Gemcitabine (8) | 40 mg/kg; q3d × 4 | 701.3 | 72.9 | 37.40 |
| BNC-1 + Gem | Combine both | 140.8 | 21.1 | 87.43 |
| (8) | | | | |

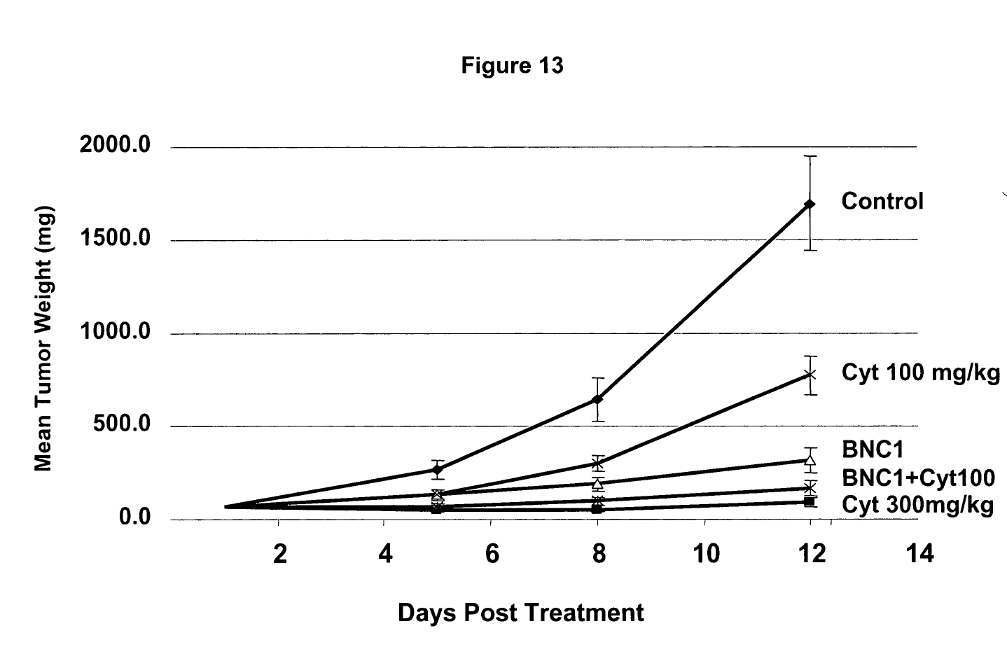

Similarly, in the experiment of FIG. 13, BNC-1 (20 mg/ml) was delivered by sc osmotic pumps (model 2002, Alzet Inc.) at 0.5 μl/hr throughout the study. Cytoxan (q1d×1) was injected at 100 mg/kg (Cyt 100) or 300 mg/kg (Cyt 300). The results again shows that BNC-1 is a potent anti-tumor agent when used alone, and its effect is additive with other chemotherapeutic agents such as Cytoxan. The result of this study is listed in the table below:

| | | Final Tumor | | |
|---|---|---|---|---|
| Group | | weight | | % |
| (Animal No.) | Dose/Route | Day 22 (Mean) | SEM | TGI |
| Control (10) | Vehicle/i.v. | 1697.6 | 255.8 | — |
| BNC-1 (10) | 20 mg/ml; s.c.; C.I. | 314.9 | 67.6 | 81.45 |
| Cytoxan300 (10) | 300 mg/ml; ip; qd × 1 | 93.7 | 24.2 | 94.48 |
| Cytoxan100 (10) | 100 mg/ml; ip; qd × 2 | 769 | 103.2 | 54.70 |
| BNC-1 + | Combine both | 167 | 39.2 | 90.16 |
| Cytoxan100 (10) | | | | |

In yet another experiment, the anti-tumor activity of BNC-1 alone or in combination with Carboplatin was tested in A549 human non-small-cell-lung carcinoma. In this experiment, BNC-1 (20 mg/ml) was delivered by sc osmotic pumps (model 2002, Alzet Inc.) at 0.5 μl/hr throughout the study. Carboplatin (qldxl) was injected at 100 mg/kg (Carb).

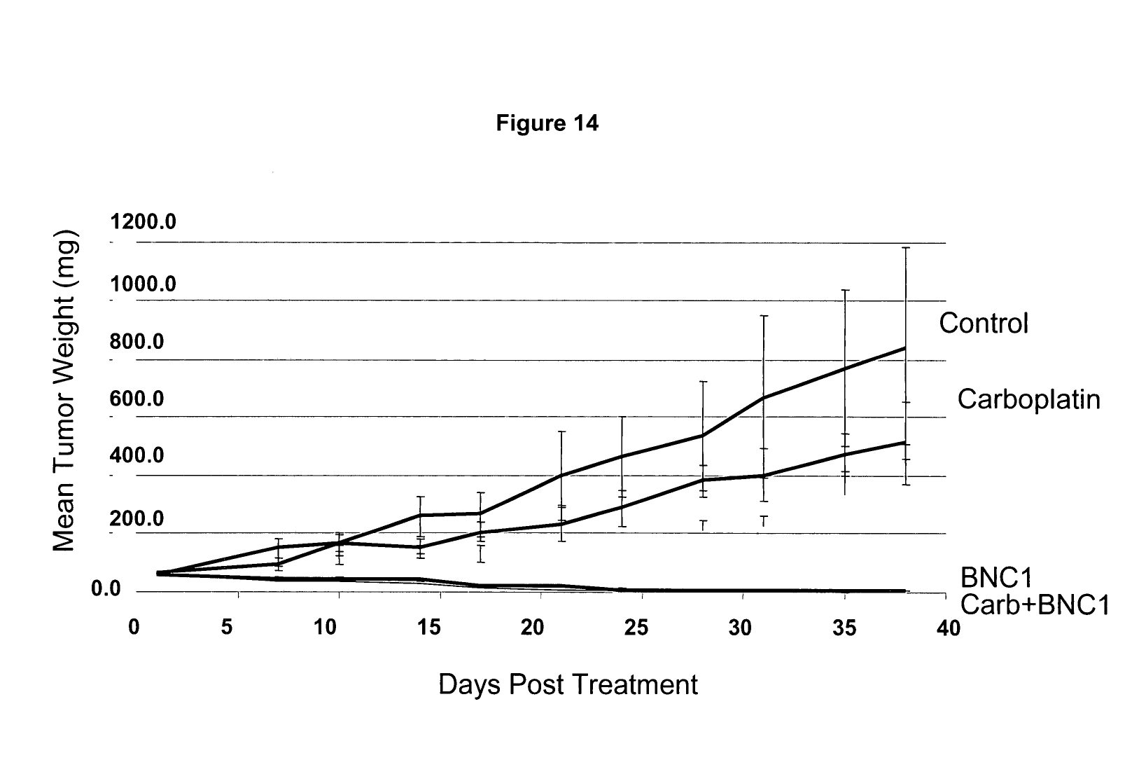

FIG. 14 confirms that either BNC-1 alone or in combination with Carboplatin has potent anti-tumor activity in this tumor model. The detailed results of the experiment is listed in the table below:

| | | % Weight | Final Tumor | | |
|---|---|---|---|---|---|
| Group | | Change at | weight Day 38 | | |
| (Animal No.) | Dose/Route | Day 38 | (Mean) | SEM | % TGI |
| Control (8) | Vehicle/i.v. | 21% | 842.6 | 278.1 | — |
| BNC-1 (8) | 20 mg/ml; s.c.; C.I. | 21% | 0.0 | 0.0 | 100.00 |
| Carboplatin (8) | 100 mg/kg; ip; qd × 1 | 16% | 509.75 | 90.3 | 39.50 |
| BNC-1 + Carb (8) | Combine both | 0% | 0.0 | 0.0 | 100.00 |

Notably, in both the BNC-1 and BNC-1/Carb treatment group, none of the experimental animals showed any signs of tumor at the end of the experiment, while all 8 experimental animals in control and Carb only treatment groups developed tumors of significant sizes.

Thus the cardiac glycoside compounds of the invention (e.g. BNC-1) either alone or in combination with many commonly used chemotherapeutic agents (e.g. Carboplatin, Gem, Cytoxan, etc.) has potent anti-tumor activities in various xenographic animal models of pancreatic cancer, renal cancer, hepatic, and non-small cell lung carcinoma.

### EXAMPLE XIII

### Effect of BNC-4 Alone or in Combination with Standard Chemotherapy on Growth of Xenografted Tumors in Nude Mice

To test the ability of BNC-4 to inhibit xenographic tumor growth in nude mice, either along or in combination with a standard chemotherapeutic agent, such as Gemcitabine, Panc-1 tumors were injected subcutaneously (s.c.) into the flanks of male nude mice. After the tumors reached 80 mg in size, osmotic pumps (model 2002, Alzet Inc., flow rate 0.5 μl/hr) containing 15 mg/ml of BNC-4 were implanted sc on the opposite sides of the mice. The control animals received pumps containing vehicle (50% DMSO in DMEM). The mice treated with standard chemotherapy agent received intra-peritoneal injections of Gemcitabine at 40 mg/kg every 3 days for 4 treatments (q3d×4). Each data point represent average tumor weight (n=8) and error bars indicate SEM.

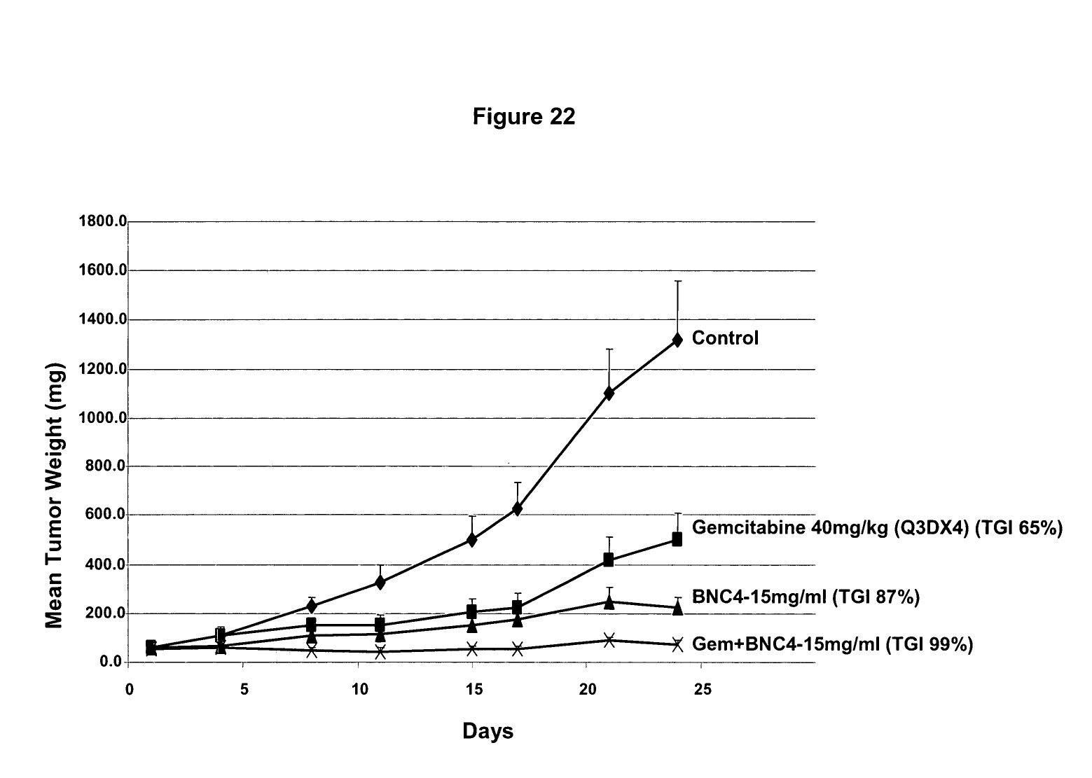

FIG. 22 indicates that, at the dosage tested, BNC-4 alone can significantly reduce tumor growth in this model. The TGI is about 87%, compared to 65% of the Gemcitabine treatment. This anti-tumor activity is additive when BNC-4 is co-administered with a standard chemotherapeutic agent Gemcitabine, with a TGI of about 99%.

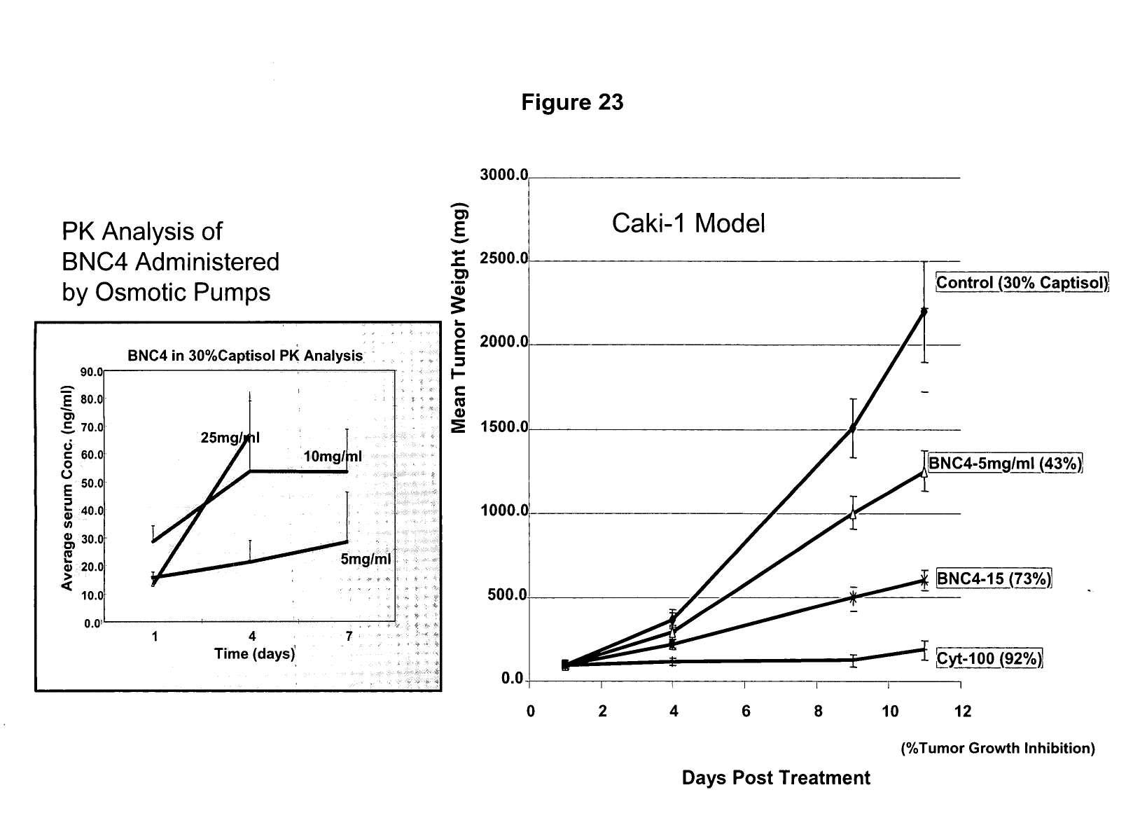

Similarly, in the experiment of FIG. 23, where renal cancer cell line Caki-1 was injected into nude mice, BNC-4 (5 or 15 mg/ml) was delivered by sc osmotic pumps (model 2002, Alzet Inc.) at 0.5 μl/hr throughout the study. Cytoxan (qldxl) was injected at 100 mg/kg (Cyt 100). The results again showed that BNC-4 is a potent anti-tumor agent when used alone (TGI of 73% and 43% for the 15 and 5 mg/ml treatment groups, respectively). As a positive control, Cytoxan achieved a 92% TGI when used alone.

Thus the cardiac glycoside compounds of the invention (e.g. BNC-4) either alone or in combination with many commonly used chemotherapeutic agents (e.g. Gem, Cytoxan, etc.) has potent anti-tumor activities in various xenographic animal models, including pancreatic cancer and renal cancer.

Pharmacokinetic studies of the BNC-4 delivered by osmotic pump were also conducted. The results of average serum concentrations of BNC-4, over the course of 1-7 days, were plotted in the left panel of FIG. 23.

### EXAMPLE XIV

### Determining Minimum Effective Dose

Given the additive effect of the subject cardiac glycosides with the traditional chemotherapeutic agents, it is desirable to explore the minimal effective doses of the subject cardiac glycosides for use in conjoint therapy with the other anti-tumor agents.

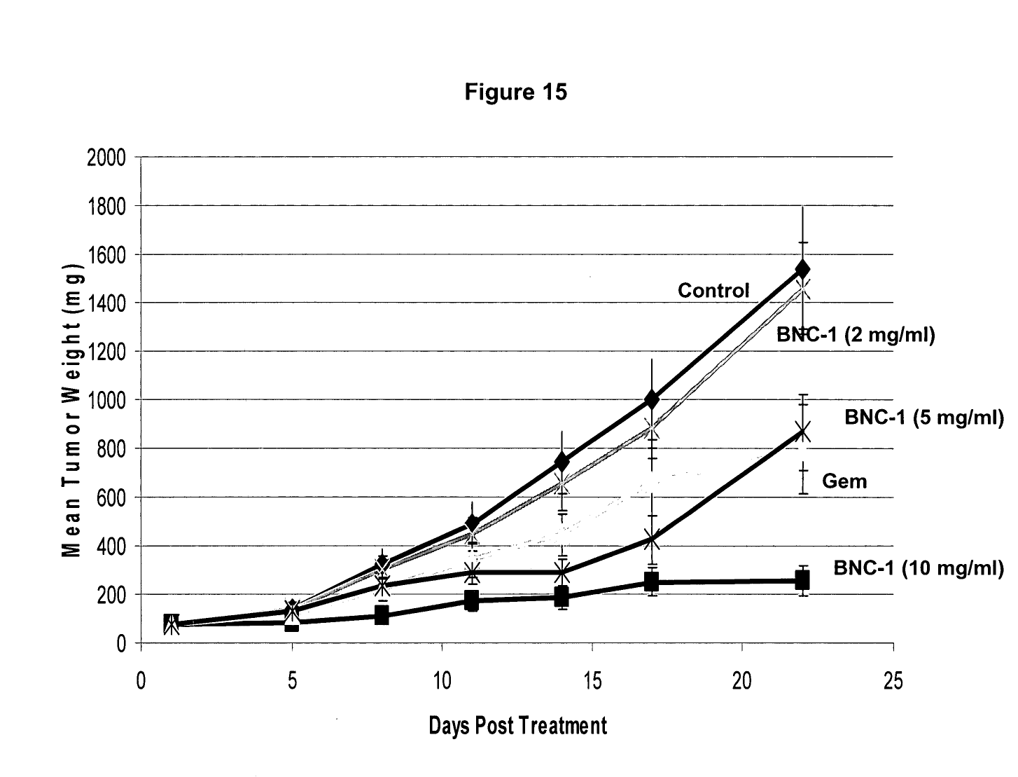

FIG. 15 shows the titration of the exemplary cardiac glycoside BNC-1 to determine its minimum effective dose, effective against Panc-1 human pancreatic xenograft in nude mice. BNC-1 (s.c., osmotic pumps) was first tested at 10, 5 and 2 mg/ml. Gem was also included in the experiment as a comparison.

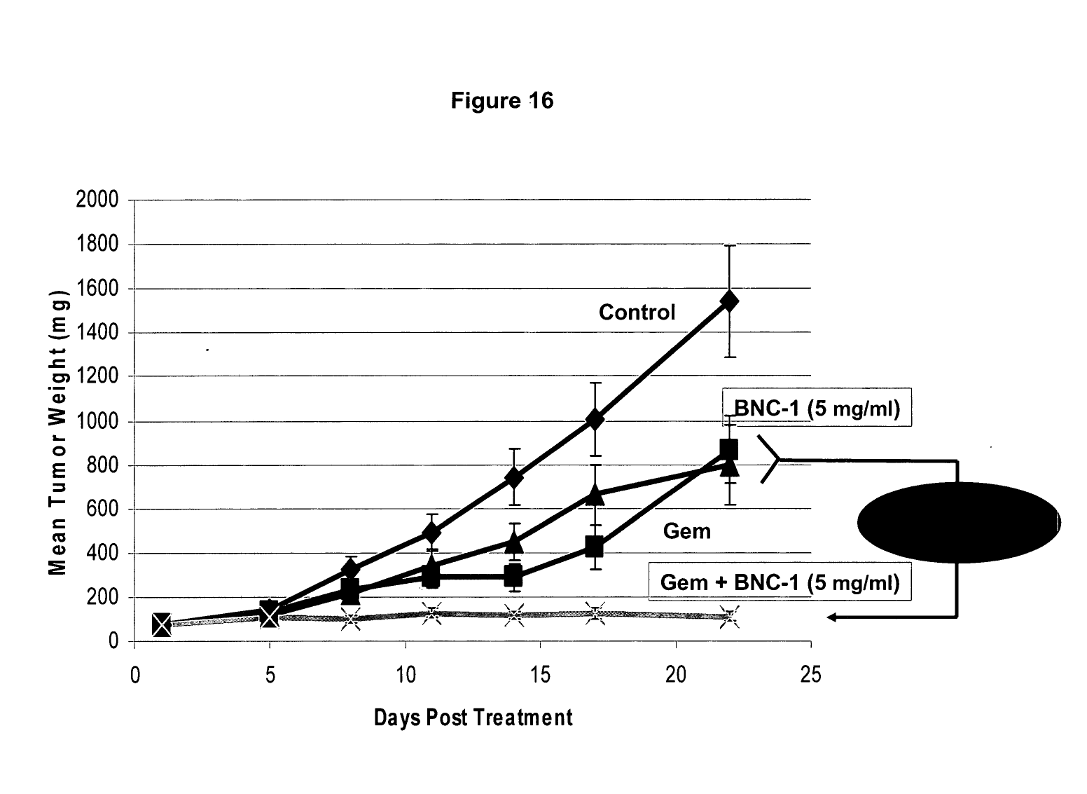

FIG. 16 shows that combination therapy using both Gem and BNC-1 produces a combination effect, such that sub-optimal doses of both Gem and BNC-1, when used together, produce the maximal effect only achieved by higher doses of individual agents alone.

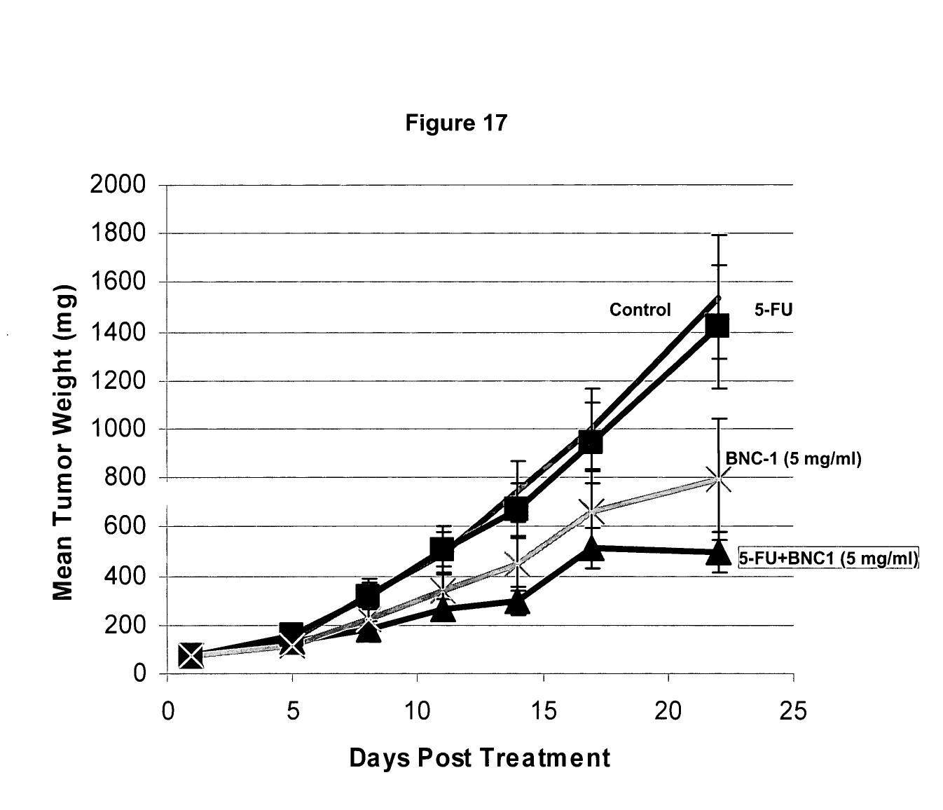

A similar experiment was conducted using BNC-1 and 5-FU, and the same combination effect was seen (see FIG. 17).

Similar results are also obtained for other compounds (e.g. BNC2) that are not cardiac glycoside compounds (data not shown).

### EXAMPLE XV

### BNC-1 and BNC-4 Inhibit HIF-1α Induced under Normoxia by PHD Inhibitor

As an attempt to study the mechanism of BNC-4 inhibition of HIF-1α, we tested the ability of BNC-1 and BNC-4 to inhibit HIF-1α expression induced by a PHD inhibitor, L-mimosone (see FIG. 6), under normoxia condition.

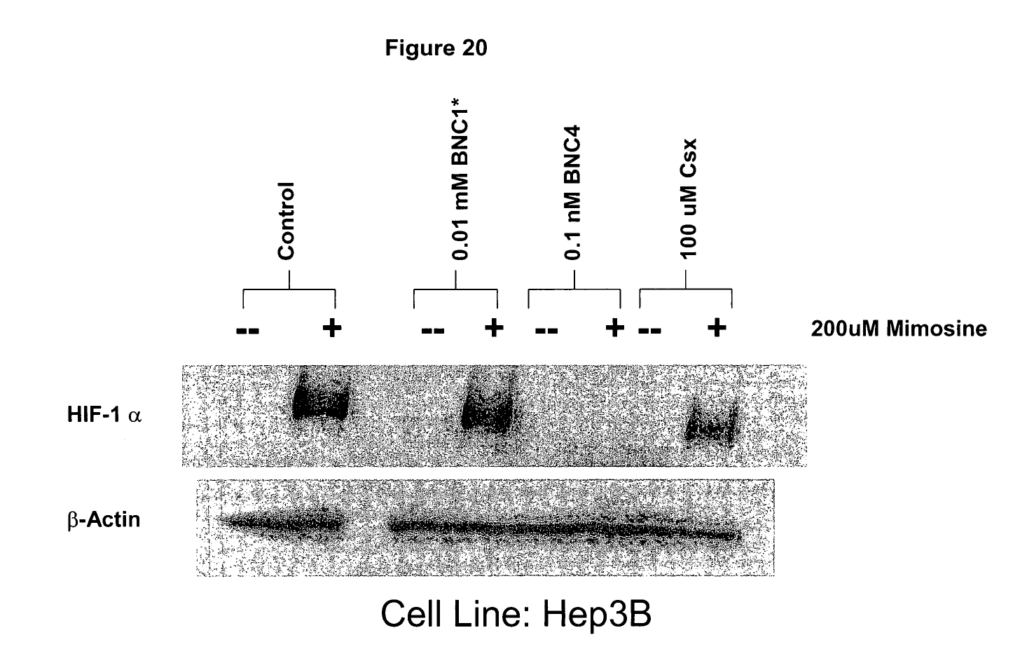

In the experiment represented in FIG. 20, Hep3B cells were grown under normoxia, but were also treated as indicated with 200 μM L-mimosone for 18 h in the presence or absence of BNC-1 or BNC-4. Abundance of HIF-1α and β-actin was determined by western blotting.

The results indicate that L-mimosone induced HIF-1α accumulation under normoxia condition, and addition of BNC-4 or BNC-1 eliminated HIF-1α accumulation by L-mimosone. At the low concentration tested, BNC-1 and BNC-4 did not appear to have an effect on HIF-1α accumulation in this experiment. While not wishing to be bound by any particular theory, the fact that BNC-4 and BNC-1 can inhibit HIF-1α induced under normoxia by PHD inhibitor indicates that the site of action by BNC-4 probably lies up stream of prolyl-hydroxylation.

### EXAMPLE XVI

### BNC-4 Inhibits Na⁺/K⁺-ATPase Activity and Has Anti-HIF/Anti-Proliferative Activity

To determine whether there is a correlation and hence validate that the observed anti-HIF/anti-Proliferative activity effects are due to an on target inhibition of Na⁺/K⁺-ATPase activity by BNC-4 and its related compounds, we measured the inhibition of Na⁺/K⁺-ATPase by BNC-4, its closely related compound BNC-151, and the aglycone BNC-147.

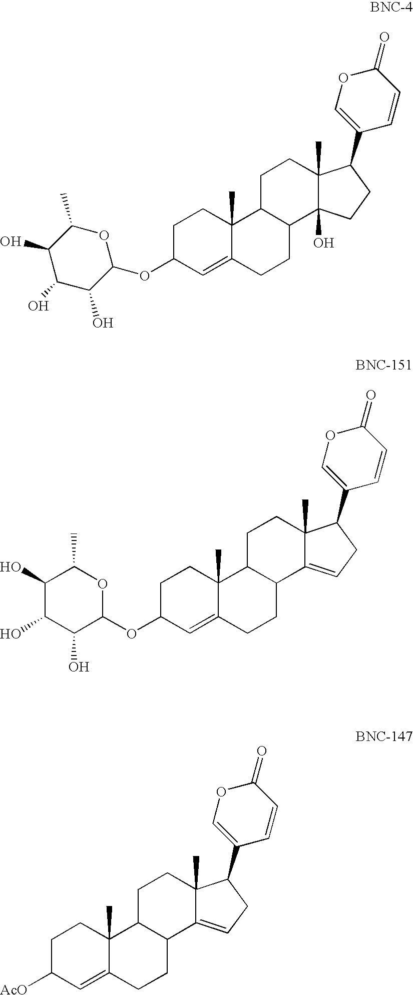

The results indicates that BNC-4 is about 10-times more potent than BNC-151, with an IC50 of about 130 nM (compared to 1380 nM for BNC-151 and 65,000 nM for BNC-147).

BNC-4 is even more potent in inhibiting cancer cell proliferation. In an anti-proliferation assay measuring % MTS activity in the A549 cell line, the IC50 for BNC-4 is only about 2.1 nM (compared to that of 260 nM for BNC-151, and 11500 nM for BNC-147).

Western blot using anti-HIF-1α antibody showed that BNC-4 completely inhibits HIF-1α expression at both 1 uM and 0.1 μM. Significant inhibition of HIF-1α expression was also observed for BNC-151 at 1 μM, and 0.1 μM to a lesser extent.

### EXAMPLE XVII

### The Bufadienolides are more Potent in Activity than the Cardenolides

To validate correlation between Na⁺/K⁺-ATPase activity and identify best in class, in terms of anti-prolferative activity we conducted experiments to profile various known cardiac glycosides and different analogues of BNC-4 for their anti-prolerafitive and anti- Na⁺/K⁺-ATPase activity. The relative activity of the bufadienolide class of cardiac glycosides was determined to be much greater then cardenolide class.

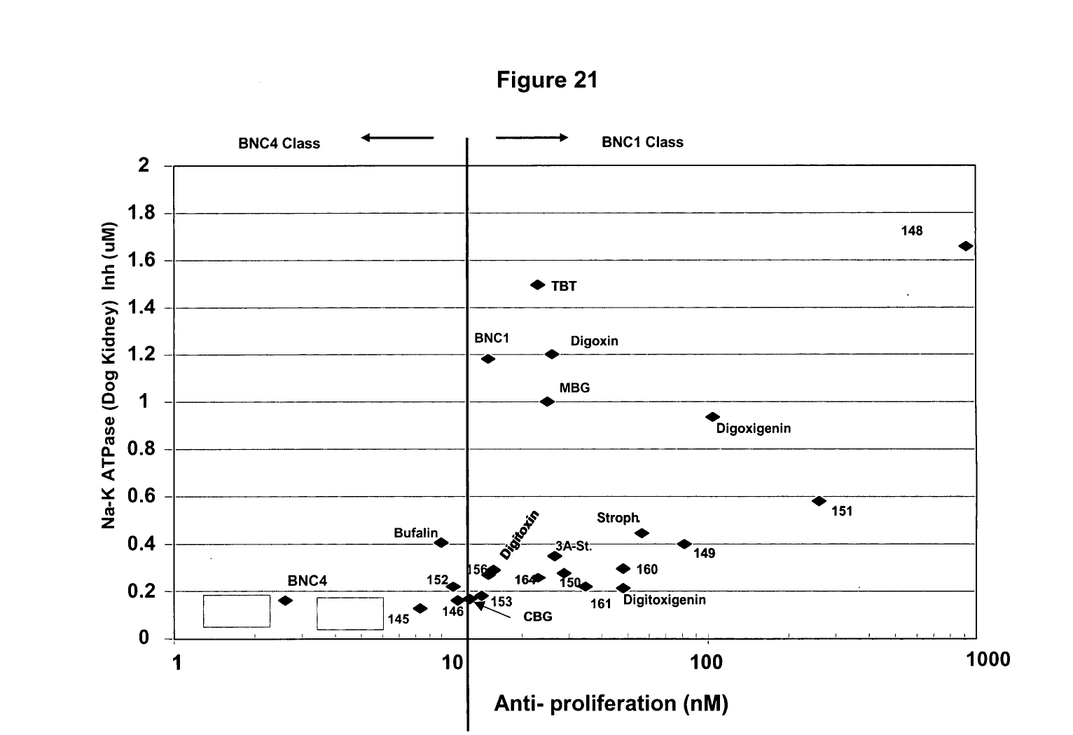

Anti-prolerafitive IC₅₀ values were determined by MTS assay using an A549 cell line. Na⁺/K⁺-ATPase inhibition IC₅₀ values were obtained using enzyme preparation from dog kidney (Sigma). The results of these assays were summarized in FIG. 21.

It is apparent that the a correlation between Na⁺/K⁺-ATPase activity and anti-proleferative activity is present and that the bufadienolides are generally more potent than the cardenolides as Na⁺/K⁺-ATPase inhibitors and anti-proliferation agents.

The subject bufadienolides and aglycones thereof preferably have anti-proliferation IC₅₀ of less than about 500 nM, more preferably less than about 11 nM, 10 nM, 5 nM, 4, nM, 3 nM, 2 nM, or 1 nM.

The subject bufadienolides and aglycones thereof preferably have anti-Na/K-ATPase IC₅₀ of less than about 0.4 μM, more preferably less than about 0.3 μM, 0.2 μM, or 0.1 μM.

In contrast, the subject cardenolides generally have anti-proliferation IC₅₀ of about 10-500 nM (see FIG. 21).

Experiments were also conducted to demonstrate that there is an inverse correlation between target Na⁺/K⁺-ATPase levels in cancer cell lines, and the anti-proliferative activity of the cardiac glycosides (e.g., bufadienolides, such as BNC-4).

Specifically, the anti-proliferative IC₅₀ values were determined for 11 established cell lines from various cancers, namely A549, PC-3, CCRF-CEM, 786-0, MCF-7, HT-29, Hop 18, SNB78, IGR-OV1, SNB75, and RPMI-8226. These cancer cell lines have different amounts of isoform-1 and isoform-3 of Na/K-ATPase, and the total amount of the two isoforms in each cell line were determined by quantitating the mRNA levels of the two isoforms by real time RT-PCR (TaqMan), using fluorescent labeled TaqMan probes. The anti-proliferation IC₅₀ values were determined by MTS assay as above. The results were plotted (total level of target Na⁺/K⁺-ATPase mRNA v. IC₅₀).

The measured IC₅₀ values range between 3.5-18.2 nM, while the total mRNA levels varied between 261-1321 arbitrary units. And the correlation coefficient (R) value was determined to be −0.73.

### EXAMPLE XVIII

### Dosage Forms and Pharmacokinetic Studies for BNC-4/Proscillaridin

This example provides a typical pharmacokinetic study for one exemplary bufadienolide cardiac glycosides—proscillaridin. Similar studies may be carried out for any of the other cardiac glycosides that can be used in the instant invention.

A. Therapeutic Use and Approval Status:

Proscillaridin was first introduced in Germany in 1964 by Knoll AG (now Abbott) (Talusin®), by Sandoz (now Novartis) (Sandoscill®), and other companies as an alternative to Ouabain (g-Strophanthin) and Digoxin/Digitoxin for acute and chronic therapy of congestive heart failure. Since then the substance was approved in Australia, Austria, Finland, France, Greece, Italy, Japan, the Netherlands, New Zealand, Norway, Poland, Portugal, Russia (and other countries of the former Soviet Block), Spain, Sweden, Switzerland, and several countries in South America (e.g. Brazil, Argentina). However, Proscillaridin has never been approved for any indications in the U.S.

Trade names include Caradrin, Cardimarin, Cardion, Encordin, Neo Gratusimal, Procardin, Proscillaridin, Prosiladin, Protosin, Proszin, Sandoscill, Scillaridin, Scillarist, Stellarid, Talusin, Theocaradrin, Theostellarid, Theotalusin, Tradenal, Tromscillan, etc. Thus “Proscillaridin” as used herein includes all forms of these compounds and their minor variants.

Numerous scientific papers have been published in the literature on the chemistry, pharmacology, uses and usefulness of Proscillaridin and related compounds. However, with the advent of ACE-inhibitors and latergeneration beta-blockers, the therapeutic use of cardiac glycosides has been on the retreat, only Digoxin being still widely prescribed.

B. Cardiac Pharmacology:

Basically, Proscillaridin shares its cardio active action with other cardiac glycosides such as Digoxin or Ouabain. The contraction of the myocardium is increased (positive inotropic effect), frequency and electric stimulus transduction are decreased (negative chronotropic effect); at low doses the transduction threshold is decreased, while it increases at higher doses. The latter effect can lead to heterotopic stimuli such as extra-systoles and arrhythmia, which are part of the pattern of symptoms appearing at intoxication levels.

The molecular mechanism of the cardiac action of Proscillaridin is more-or-less identical to that of the other cardiac glycosides, and centers on the modulation/inhibition of the sarcoplasmic Na/K-ATPase ion pump. This trans-membrane protein exchanges three cytosolic sodium ions for two extra-cellular potassium ions at the expense of ATP. The Na⁺/K⁺-ATPase protein consists of two subunits (α and β), which are assembled on demand together with a third (γ) subunit to form the functional enzyme complex. The α- and β-subunits come in different isoforms (so far 4 isoforms have been described for the α-subunit, and 3 for the β-subunit), which allows for a large variety of Na⁺/K⁺-ATPase isoforms to exist. The different variations are tissue-specific, and show different affinities towards cardiac glycosides. This explains the specific high sensitivity of myocardial muscle fibers and adrenergic nerve cell membranes towards cardiac glycosides.

For example, based on Western blot analysis, the alphal isoform of Na⁺/K⁺-ATPase is constitutively expressed in most organisms tested, including brain, heart, smooth intestine, kidney, liver, lung, skeletal muscle, testis, spleen, pancrease, and ovary, with the most abundant expression observed in brain and kidney. The alpha2 isoform is largely expressed in the brain, muscle, and heart. The alpha3 isoform is rich in the CNS, especially the brain. The alpha4 isoform appears to be specific for the testis. There exist two binding sites for cardiac glycosides among the Na⁺/K⁺-ATPase α-subunits: a high-affinity/low-density site, and a low-affinity/high-density site. About 25% of all binding sites on ventricular muscle cells are of the high affinity type (Akera T et al. 1986). Very small amounts of cardiac glycosides (e.g., Ouabain) stimulate rather than inhibit sodium pump action, presumably by interacting with the high-affinity binding sites (Gao et al. 2002). These binding events trigger a variety of signal cascades involved in cellular growth by controlling the binding of the α-subunit to Caveolin-1, an essential protein for intra-cellular signal-transduction and vesicular trafficking (Wang H et al. 2004). At higher local concentrations of cardiac glycoside also the low-affinity binding site becomes involved, and the overall enzyme exchange rate diminishes. This results in a net loss of intracellular potassium, leading to a sodium imbalance, which is in turn offset by calcium influx by way of the Na⁺/Ca²⁺-exchanger. The increased concentration of intracellular calcium leads to a higher contractility of the myocardial cells, resulting in a stronger and more complete contraction of the heart muscle.

In a comparative study of therapeutically used cardiac glycosides the order of Na⁺/K⁺-ATPase-inhibition was Ouabain<Digoxin<Proscillaridin, making Proscillaridin one of the most potent modulators of the sodium pump (Erdmann E 1978). (For a comprehensive overview on the molecular- and clinical pharmacology of Cardiac Glycosides in general, and Digitalis Glycosides in particular, see: Karl Greeff (Ed.) “Cardiac Glycosides”, 2 Vols., Springer Verlag, 1981; and: Thomas Woodward Smith (Ed.) “Digitalis Glycosides”, Grune & Stratton 1986).

C. Anti-Cancer Indication and Mechanism-of-Action:

Proscillaridin A is a potent cytotoxic agent against a panel of 10 cancer cell lines, with a median IC₅₀ of about 23 nM (compared with 37 nM for Digoxin, and 78 nM for Ouabain).

While not wishing to be bound by any particular theory, the theory that cardiac glycosides, such as Proscillaridin, exerts their effect through acting on the sodium pump (Na⁺/K⁺-ATPase) is an attractive model for explaining the anti-cancer activity of cardiac glycosides in general and Proscillaridin in particular.

On one hand, there is ample evidence that increased intracellular calcium concentrations disturb the action potential across the mitochondrial membrane, increasing the uncontrolled proliferation of reactive oxygen species (ROS) and triggering apoptotic cascades. On the other hand, glycoside binding to the Na/K-ATPase is by itself a signaling event, inducing the Src-EGFr-ERK pathway, activating protein tyrosine phosphorylation and mitogen-activated protein kinases (MAPK), and increasing the production of ROS (see, for example: Tian J, Gong X, Xie Z. 2001. Ferrandi M et al. 2004).

Applicants have found for the first time that Ouabain and, to an even larger degree, BNC-4 (Proscillaridin) induce a signal that prevents cancer cells to respond to hypoxic stress through transcriptional inhibition of Hypoxia Inducible Factor (HIF-1α) biosynthesis. This may form the basis of the observed anti-cancer activity of cardiac glycosides, such as Proscillaridin, and their aglycones.

While not wishing to be bound by any particular theory, cancer cells of solid tumors are poorly vascularized, and, as a consequence, permanently exposed to sub-normal oxygen levels. As a response, they over-produce HIF. HIF1-α functions as an intracellular sensor for hypoxia and the presence of ROS. In normoxic cells, HIF-1α is continuously degraded by oxidative hydroxylation involving the enzyme proline-hydroxylase. Lack of oxygen prevents this degradation, and allows HIF to be transformed into a potent nuclear transcription factor. Its multi-valency makes it a central turn-on switch for the transcription of a wide variety of growth factors and angiogenic factors that are essential for malignant survival, growth and metastasis. By inhibiting HIF-1α biosynthesis, BNC-4 prevents cancer cells from producing these factors, and hence from proliferating, invasion, and metastasis.

Since in cancer cells, the distribution and combination of isoforms of the sodium pump, and hence the sensitivity towards cardiac glycosides is often dramatically altered, treatment with BNC-4 and its analogs allow cancer-specific molecular intervention with minimum effects on healthy tissues (Sakai et al. 2004, and references cited therein).

D. Pharmacokinetics:

a) Absorption:

Orally dosed Proscillaridin is rapidly, yet incompletely absorbed. The reported values range from 7 to 40%, with an accepted median at about 20%. These values were determined, however, with simple oral formulations (hydroalcoholic solutions or tablets), comparing i.v. and oral doses necessary to achieve pulse normalization in tachycardic patients (Hansel 1968; Belz 1968).

It has become evident that exposing Proscillaridin to stomach acid causes substantial decomposition (Andersson K E et al. 1976, 1975b; Einig H 1976). Thus the invention provides special dosage forms for certain patients, such as those taking antacids routinely, because in these patients, there is decreased stomach acid production, resulting in up to 60% higher absorption of Proscillaridin (Andersson K E 1977c). Proper adjustments are made in these special dosage forms to ensure the same final serum concentration effective for cancer treatment.

In other embodiments, the subject oral formulations mitigates this acid instability by including an acid-resistant coating, such as an enteric coating. With such a dosage form, absolute bioavailability is increased to about 35%. These data show that orally dosed Proscillaridin is being absorbed and distributed at a significant and measurable level, and behaves in this respect not differently from many other successful drugs with rapid first-pass metabolism (Pond S M, Toser T N 1984).

b) Distribution

After oral administration, peak blood concentrations of unconjugated Proscillaridin are reached after 15-30 minutes (Belz G G et al. 1973, 1974; Andersson K E et al. 1977a). However, the absolute value of measurable unconjugated drug reflects only 7% of the administered quantity, most likely a consequence of the formulation used in the experiment, the instability in gastric juices, and extensive first-pass metabolism (conjugation) in the gut wall (see below). The striking difference between portal and peripheral blood indicates a rapid tissue distribution.

Monitoring blood levels at 10-minutes intervals reveals a second, longer-lasting peak at about 1 hour: at this time, equilibrium between free and bound drug has been reached. Measuring of plasma concentrations over a longer period reveals that a third peak is reached at about 10 hours after dosing (Belz GG et al. 1974). This multi-phasic distribution is characteristic for entero-hepatic recycling of cardiac glycosides: the conjugates are excreted into the intestine, cleaved by the local bacteria, and the de-conjugated drug is re-absorbed (Andersson K E et al 1977b).

For clinical purposes it is important to know that optimal therapeutic plasma levels (EC) can be achieved with a single oral dose of 3.5 mg in as short as 30 minutes, and steady state is reached after 48 to 72 hours by continuing doses of 1.0 to 1.5 mg/d (Heierli C et al. 1971)(see “Posology” below). At this level about 85% of the substance is bound to plasma protein (Kobinger W, Wenzel W 1970).

Intravenous injection of 0.9 mg produced a plasma concentration of 1.09 ng/mL (measured by 86Rb-uptake; Belz G G et al. 1974a), giving a Volume-of-Distribution (VD) of 562 liters; this comparatively large value indicates an extensive tissue distribution typical for cardiac glycosides (compare to VD for Digoxin −650 liters).

In this context, it is important to note the differences in measurable plasma drug levels depends on the method used. In contrast to the values obtained by ⁸⁶Rb-uptake, radio-immunoassays of plasma samples from 12 healthy individuals receiving 2×0.5 mg Talusin for 8 days gave a median Cₘₐₓ of 23.5±2.6 ng/mL, and Tₘₐₓ of 0.8±0.5 hours, with a median AUC of 385.0±43.6 ng/mL×h (Buehrens K G et al. 1991). While the former method measures only un-conjugated glycoside, which has to be extracted with dichloromethane prior to measurement, RAIs and ELISAs can be applied directly to plasma samples and measure free and conjugated drug together. Considering that the conjugates are still bioactive, the latter methods deliver probably a more indicative picture for the present indication. Unless specifically indicated otherwise, the serum concentration used herein refers to the total concentration of the subject cardiac glycosides, including conjugated/unconjugated forms bound or unbound by serum proteins.

c) Metabolism and Excretion:

For Proscillaridin, the total level of metabolism is >95%. In the stomach the glycosidic linkage is hydrolytically cleaved to a large extent, depending on the formulation used. Nevertheless, the de-glycosylated aglycone (e.g., Scillarenin for Proscillaridin and Scillaren) has a similar biological activity, and is also absorbed by the gut. During passage through the gut wall and subsequent liver passage, the substance becomes conjugated to glucuronic acid and sulfuric acid, and is secreted predominantly with bile. Subsequent de-conjugation by intestinal bacteria leads to partial re-absorption, resulting in the bi-phasic excretion profile mentioned above (Andersson K E et al. 1977b). Oxidative metabolism by P450 enzymes is much less pronounced, leading again to cleavage of the sugar linkage. Greater than 99% of the drug and its metabolites are excreted by the bile, while less than 1% of unchanged Proscillaridin is excreted by the kidneys. This independence of excretion from renal function makes the drug especially valuable for the treatment of patients with acute or chronic kidney disease, such as (refractory) renal cancer.

d) Plasma Concentration and Clearance:

The median plasma half-life (T_{1/2}) of Proscillaridin range from 23 to 29 hours in healthy individuals, and up to 49 hours median in cardiac patients (Belz G G, Brech W J 1974; Belz G G, Rudofsky G et al. 1974; Bergdahl B 1979), with daily clearance being ˜35%. The latter value is very different from those for Digitalis glycosides, which makes Proscillaridin the preferred drug when good control and quick dose adjustment to negative effects is essential.

Because the drug is almost entirely excreted through the bile, impaired kidney function has no influence on clearance (Belz G G, Brech W J 1974).

The measurements of therapeutic plasma levels at steady state vary, depending on the analytical methodology used (see above). Measuring the uptake of the Rubidium isotope ⁸⁶Rb by erythrocytes exposed to plasma gives values of circulating un-conjugated un-bound Proscillaridin ranging from 0.2 to 1.0 ng/mL (Cₘₐₓ) (Belz G G et al. 1974a); radio-immune assays on the other hand, do not distinguish between un-conjugated and conjugated or plasma-bound vs. free drug, and show levels between 10 and 30 ng/mL. It is probable that therapeutic action is also produced by the plasma-bound drug, and, albeit probably to a lesser extent, by the conjugates, as has been shown for Digoxin (Scholz H, Schmitz W 1984). Conjugate concentrations in blood plasma reached almost 20 ng/mL after a single oral dose of 1.5 mg Proscillaridin (Andersson et al 1977a).

Nevertheless, the median effective concentration (EC₅₀) of free Proscillaridin for cardiac indications is about 0.8 ng/mL (Belz G G et al. 1974c), which can be maintained by a median oral dosage of 0.9 mg/d (Loeschhom N 1969). The median effective concentration (EC₅₀) of free Proscillaridin for the subject cancer indications is about 1.5 to 3 times that of cardiac indications, or about 1.2-2.5 ng/mL of free (unbound, unconjugated) Proscillaridin.

e) Posology:

In cardiac patients, at doses of 1.5 mg/d, steady states of therapeutic plasma levels are reached after 3 to 5 days (loading-to-saturation) with very few side-effects reported. The duration of cardiac action after saturation lies between 2 and 3 days. The optimal therapeutic level for cardio-vascular indications (ED_{p.o.}) was determined to be close to 5 mg by measuring the amount necessary to normalize tachycardia/fibrillation. Thus a one-time dose of 3.5 mg/d, followed by maintenance doses of 1.5 mg for two days and I mg/d thereafter can achieve this level in about 60 hours (Heierli C. et al. 1971; Hansel 1968). Belz determined the optimal median maintenance dose to be 1.86 mg (Belz 1968).

A more conservative approach achieves therapeutic levels by saturation-dosing over 4-5 days with 1.5 mg/d, followed by doses of 0.5-1.5 mg/d depending on individual tolerances. Because of the rapid excretion kinetics, slow ramping-up towards saturation doses (as it is usual practice with Digoxin) is not necessary. In cases of increased need for glycoside effect, daily doses of 2.0 or even 2.5 mg have been used in cardiac patients.

For clinical purposes in the cardiovascular field, the indirect determination of optimal circulating concentrations is more practical: the substance is injected intravenously at tolerable intervals up to a total dosage that produces the desired effect (in the case of Proscillaridin this could be for example the disappearance of atrial fibrillation); subsequently, the drug is given orally at sub-toxic doses until the same effect is achieved. This dose is the Effective oral Dose (EDP.O.), which for Proscillaridin can go as high as 8.5 to 13.1 mg (total loading dose), depending on the speed of administration (2.25 mg/d for 4 days vs. 1.5 mg/d for 9 days), and from 0.65 up to 1.8 mg for maintenance of therapeutic levels (see for example: Gould L et al. 1971, or Bulitta A 1974).

For the present cancer indication, the effective oral dose is generally about 1.5-3 times for the cardiac indication. It is important to notice that, comparison studies between patients with cardiac insufficiency and cardiologically-normal individuals showed clearly that the latter have a much better tolerance for Proscillaridin before the onset of typical glycoside intoxication symptoms, changes in ECG, and metabolite profile (Gebhardt et al. 1965; Doneff et al. 1966); doses of up to 3.5 mg/d were well tolerated in cardiologically-normal individuals (Heierli et al. 1971).

However, in light of the often diminished body weight of cancer patients, and the fact that decreased stomach acid produces higher plasma concentrations, careful monitoring for appearance of toxic side effects at rapid saturation dosing will be essential in patients that fit these descriptions.

Toxicology:

The LD₅₀ p.o. in rats is reported as 0.25 mg/Kg in adult, and as 76 mg/Kg in young animals (female), making Proscillaridin about half as toxic as Digitoxin (0.1 mg/Kg/adult) (Goldenthal E I 1970). Rodents, however are bad toxicity indicators for cardiac glycosides because of their pronounced insensitivity towards this particular compound class (with the exception of Scillirosid, which is actually used as a rodenticide).

Intravenous toxicity in cats was determined to be 0.193 mg/Kg, positioning Proscillaridin in between Ouabain (0.133 mg/Kg) and Digoxin (0.307 mg/Kg). Duodenal administration, however, reverses this order, probably due to metabolic transformation during absorption by the gut wall. The values are: 5.3 mg/Kg for Ouabain, 1.05 mg/Kg for Proscillaridin, and 0.78 mg/Kg for Digoxin (Lenke D, Schneider B 1969/1970). Similar values were found in guinea pigs (Kurbjuweit H G 1964; Kobinger W. et al. 1970). These toxicology data helps to guide skilled artisans to set the upper limit dosage for the treatment of refractory cancers.

a) Acute Toxicity:

Proscillaridin exhibits about half the toxicity of Ouabain (Melville KI et al. 1966). The relatively wide therapeutic window of the compound in comparison to Ouabain or Digoxin is due to a combination of plasma-protein binding and rapid clearance (Kobinger W. et al. 1970); nevertheless, doses above 4 mg p.o./d in healthy individuals produce the for cardiac glycosides typical intoxication symptoms (nausea, headaches, seasickness, cardiac arrhythmias, bradycardia, extrasystoles).

However, the great advantage of Proscillaridin over other cardiac glycosides lies in the rapid clearance of the drug, so that toxic symptoms disappear very quickly after dosing is discontinued.

b) Chronic Toxicity:

Proscillaridin is still prescribed in Europe for the long-term medication of various cardiac illnesses. Patients take up to 1.5 mg per day without any negative side effects. The longest clinical and post-clinical observation of patients taking Proscillaridin was published in 1968: 1067 patients were observed for up to 3 years after their initial dose, which was often a switch from Digitalis (Marx E. 1968). Of these only 0.7% developed negative side effects to such an extent that they had to be taken off the treatment. Upon reviewing the clinical safety data of Proscillaridin in a total of 3740 patients, Applicants found that none of these cases noted any long-term or late-appearing chronic toxicity.

c) Side Effects:

In healthy volunteers, 1.5 mg daily for 20 days produced no negative side effects (Andersson K E et al. 1975). Changes in color vision (Gebhardt et al. 1965) and other symptoms typical for Digitalis intoxication disappeared in patients after the switch from Digitalis to Proscillaridin. The only remarkable side effects that appear in almost all clinical reports at a level of 5% average are nausea, seasickness, headache, vomiting, stomach cramps and diarrhea (in order of decreasing frequency); very few patients develop cardiac arrhythmias or bradycardia. In most cases, these symptoms were of a transient nature, and could be controlled by temporarily lowering the administered dose. It must be mentioned, however, that in most instances the individuals under observation were very ill cardiac patients, which are known to have a higher sensitivity towards cardiac glycoside action and side-effects than cardiologically-healthy individuals.

In the clinical trial results study below, a small percentage (about 6.3%) of the patients also exhibited certain side-effects, the most negative symptoms being: nausea, stomach irritation, sea-sickness, diarrhea, cardiac arrhythmia, bradycardia, and extra-systoles. However, these symptoms are mostly transient. In >95% of the reported cases, therapy could be resumed after a brief hiatus.

d) Interactions with Other Drugs:

Possible negative interactions with other drugs are the same for Proscillaridin as with other cardiac glycosides such as Digoxin or Digitoxin. The corresponding precautions can be taken from the respective monographies in the Physician's Desk Reference. Coprescription of anti-hypertensives, vasodilators and diuretics are quite common with Proscillaridin. The molecular mechanism of action involves modulation of the Na/K-ATPase ion-pump (see above paragraph), resulting in a net loss of intracellular potassium and an increase of this ion in the plasma. Therefore, the possibility of hyperkalemia, especially during the loading phase of the treatment with Proscillaridin, warrants careful monitoring of electrolyte levels. Thus in certain embodiments, the method of the invention include a further step of monitoring electrolyte levels in patients subject to the treatment to avoid or ensure early detection of hyperkalemia and other associated side-effects.

On the other hand, when diuretics are being used concomitantly, the danger of alkalosis exists, and K and Cl must eventually be replaced. Quinidine, used as an anti-arrhythmic, diminishes hepatic excretion of Proscillaridin, and blood plasma levels might rise accordingly.

Cardiac glycosides, in conjunction with vasodilators and diuretics, have shown beneficial effects on myocardial failure scenarios in cancer patients after radiation or doxorubicin therapy (for example: Haq M M et al. 1985; Schwartz R G et al. 1987; Cordioli E et al. 1997).

Clinical Safety

Clinical safety of the subject cardiac glycosides, particularly safety in severely ill patient populations, including cancer patients, has also been evaluated.

Applicants have reviewed clinical trial results compiled from 47 clinical studies from the years 1964 to 1977. These studies describe a total of 3740 patients on Proscillaridin A treatment over an observation period of as long as 3 years. The studies were especially analyzed for the observation of acute or chronic negative side effects in relation to the initial diagnoses present at commencement of the medication.

Also noted are any concomitant medications to detect any incompatibilities. In most of the analyzed studies the patient population consisted of seriously ill individuals: besides severe heart conditions, many patients had concomitant diagnoses ranging from diabetes-mellitus, liver cirrhosis, hypertension, pulmonary and/or hepatic edema, bronchial emphysema, kidney failure, gastritis, stomach ulcers, and/or severe obesity.

Despite the general poor condition of these patients, and in respect to the present study, it is important to notice that the large majority of these severely ill patients tolerated Proscillaridin A very well. Proscillaridin A was well-tolerated at ˜1.5 mg/d in these cardiac patients, and up to about 3.5 mg/d in cardiologically normal individuals.

For example, in one of the studies reviewed (Bierwag K 1970), Proscillaridin was given to non-cardiac patients as a prophylactic to prevent occurrence of cardiac complications during and after impending surgery. The 50 patients described ranged in age from 50 to 83 years. The majority were cancer patients with the following diagnoses:

Gall bladder carcinoma

Papillary carcinoma

Stomach carcinoma

Colorectal adenocarcinoma

Mamma carcinoma

The patients received 0.25 to 0.5 mg/d intra-venously for four days before surgery and 0.25 mg/d during the four following days; they were then switched to an oral dose of 0.75 to 1.5 mg/d.

Considering the pharmacokinetic characteristics of Proscillaridin described above, 0.5 mg/d i.v./4 d is equivalent to an oral dose for loading of roughly 2.5 mg/d for three days, or 1.8 mg/d for 4 days. This dose was well tolerated by all cancer patients with no appearance of either gastrointestinal or cardiac side effects.

### EXAMPLE XIX

### Estimation of Therapeutic Index from Steady State Delivery of Compounds in Mice

To estimate the therapeutic index of the subject cardiac glycosides, we measured the therapeutic serum concentrations of the subject cardiac glycosides (e.g., BNC-1 and BNC-4) required to achieve greater than 60% tumor growth inhibition (TGI), and the corresponding toxic serum concentrations for these cardiac glycosides.

For BNC-1, the therapeutic serum concentration required to achieve >60% TGI is about 20±15 ng/ml, while the toxic serum concentration at day 1 is about 50±21 ng/ml. Therefore, the therapeutic index (toxic concentration/therapeutic level) for BNC-1 is about 2.5.

In contrast, for BNC-4, the therapeutic serum concentration required to achieve >60% TGI is about 48±23 ng/ml, while the toxic serum concentration at day I is about 518±121 ng/ml. Therefore, the therapeutic index (toxic concentration/therapeutic level) for BNC-4 is about 10.79. This suggests that BNC-4 and other bufadienolides and aglycones thereof generally have higher therapeutic index, and are preferred over the cardenolides.

All publications and patents mentioned herein are hereby incorporated by reference in their entirety as if each individual publication or patent was specifically and individually indicated to be incorporated by reference. In case of conflict, the present application, including any definitions herein, will control.

Equivalents:

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A pharmaceutical formulation comprising a Na⁺/K⁺-ATPase inhibitor in an oral dosage form and an anti-cancer agent that induces a hypoxic stress response in tumor cells, formulated in a pharmaceutically acceptable excipient and suitable for use in humans to treat a neoplastic disorder, wherein the oral dosage form maintains an effective steady state serum concentration of from about 10 to about 700 ng/mL.

2-4. (canceled)

5. A pharmaceutical formulation comprising a Na⁺/K⁺-ATPase inhibitor in an oral dosage form and an anti-cancer agent that induces a hypoxic stress response in tumor cells, formulated in a pharmaceutically acceptable excipient and suitable for use in humans to treat a neoplastic disorder, wherein the oral dosage form comprises a total daily dose of from about 2.25 to about 7.5 mg per human individual.

6-35. (canceled)

36. A pharmaceutical formulation comprising scillaren in an oral dosage form and an anti-cancer agent that induces a hypoxic stress response in tumor cells, formulated in a pharmaceutically acceptable excipient and suitable for use in humans to treat a neoplastic disorder.

37. A kit for treating a patient having a neoplastic disorder, comprising a Na⁺/K⁺-ATPase inhibitor in an oral dosage form and an anti-cancer agent that induces a hypoxic stress response in tumor cells, each formulated in premeasured doses for conjoint administration to a patient to treat a neoplastic disorder, wherein the oral dosage form maintains an effective steady state serum concentration of from about 10 to about 700 ng/mL.

38-40. (canceled)

41. A kit for treating a patient having a neoplastic disorder, comprising a Na⁺/K⁺-ATPase inhibitor in an oral dosage form and an anti-cancer agent that induces a hypoxic stress response in tumor cells, formulated in a pharmaceutically acceptable excipient and suitable for use in humans to treat a neoplastic disorder, wherein the oral dosage form comprises a total daily dose of from about 2.25 to about 7.5 mg per human individual.

42-71. (canceled)

72. A kit comprising scillaren in an oral dosage form and an anti-cancer agent that induces a hypoxic stress response in tumor cells, formulated in a pharmaceutically acceptable excipient and suitable for use in humans to treat a neoplastic disorder.

73. A method for treating a patient having a neoplastic disorder comprising administering to the patient an effective amount of a Na⁺/K⁺-ATPase inhibitor in an oral dosage form and an anti-cancer agent that induces a hypoxic stress response in tumor cells, wherein the oral dosage form maintains an effective steady state serum concentration of from about 10 to about 700 ng/mL.

74-76. (canceled)

77. A method comprising a Na⁺/K⁺-ATPase inhibitor in an oral dosage form and an anti-cancer agent that induces a hypoxic stress response in tumor cells, formulated in a pharmaceutically acceptable excipient and suitable for use in humans to treat a neoplastic disorder, wherein the oral dosage form comprises a total daily dose of from about 2.25 to about 7.5 mg per human individual.

78-107. (canceled)

108. A method for treating a patient having a neoplastic disorder, comprising administering to the patient an effective amount of scillaren in an oral dosage form and an anti-cancer agent that induces a hypoxic stress response in tumor cells.

109. Use of a Na⁺/K⁺-ATPase inhibitor in the manufacture of a medicament in an oral dosage form, for treating a patient having a neoplastic disorder, said Na⁺/K⁺-ATPase inhibitor is administered with an anti-cancer agent that induces a hypoxic stress response in tumor cells, said oral dosage form maintains an effective steady state serum concentration of from about 10 to about 700 ng/mL.

110-112. (canceled)

113. Use of a Na⁺/K⁺-ATPase inhibitor in the manufacture of a medicament in an oral dosage form, for treating a patient having a neoplastic disorder, said Na⁺/K⁺-ATPase inhibitor is administered with an anti-cancer agent that induces a hypoxic stress response in tumor cells, said oral dosage form comprises a total daily dose of from about 2.25 to about 7.5 mg per human individual.

114-143. (canceled)

144. Use of scillaren in the manufacture of a medicament in an oral dosage form, for treating a patient having a neoplastic disorder, wherein the scillaren is administered with an anti-cancer agent that induces a hypoxic stress response in tumor cells.

145. A method for promoting treatment of patients having a neoplastic disorder, comprising packaging, labeling and/or marketing a Na⁺/K⁺-ATPase inhibitor in an oral dosage form to be used in conjoint therapy for treating a patient having a neoplastic disorder with an anti-cancer agent that induces a hypoxic stress response in tumor cells, wherein the oral dosage form maintains an effective steady state serum concentration of from about 10 to about 700 ng/mL.

146-148. (canceled)

149. A method for promoting treatment of patients having a neoplastic disorder, comprising packaging, labeling and/or marketing a Na⁺/K⁺-ATPase inhibitor in an oral dosage form to be used in conjoint therapy for treating a patient having a neoplastic disorder with an anti-cancer agent that induces a hypoxic stress response in tumor cells, wherein the oral dosage form comprises a total daily dose of from about 2.25 to about 7.5 mg per human individual.

150-179. (canceled)

180. A method for promoting treatment of patients having a neoplastic disorder, comprising packaging, labeling and/or marketing scillaren in an oral dosage form to be used in conjoint therapy for treating a patient having a neoplastic disorder with an anti-cancer agent that induces a hypoxic stress response in tumor cells.

181. Use of a Na⁺/K⁺-ATPase inhibitor in the packaging, labeling and/or marketing of a medicament in an oral dosage form, for promoting treatment of patients having a neoplastic disorder, said Na⁺/K⁺-ATPase inhibitor is administered in conjoint therapy with an anti-cancer agent that induces a hypoxic stress response in tumor cells, wherein the oral dosage form maintains an effective steady state serum concentration of from about 10 to about 700 ng/mL.

182-184. (canceled)

185. Use of a Na⁺/K⁺-ATPase inhibitor in the packaging, labeling and/or marketing of a medicament in an oral dosage form, for promoting treatment of patients having a neoplastic disorder, said Na⁺/K⁺-ATPase inhibitor is administered in conjoint therapy with an anti-cancer agent that induces a hypoxic stress response in tumor cells, wherein the oral dosage form comprises a total daily dose of from about 2.25 to about 7.5 mg per human individual.

186-215. (canceled)

216. Use of scillaren in the packaging, labeling and/or marketing of a medicament in an oral dosage form, for promoting treatment of patients having a neoplastic disorder, said scillaren is administered in conjoint therapy with an anti-cancer agent that induces a hypoxic stress response in tumor cells.

217. A method for promoting treatment of patients having a neoplastic disorder, comprising packaging, labeling and/or marketing an anti-cancer agent that induces a hypoxic stress response in tumor cells to be used in conjoint therapy with a Na⁺/K⁺-ATPase inhibitor in an oral dosage form for treating a patient having a neoplastic disorder, wherein the oral dosage form maintains an effective steady state serum concentration of from about 10 to about 700 ng/mL.

218-220. (canceled)

221. A method for promoting treatment of patients having a neoplastic disorder, comprising packaging, labeling and/or marketing an anti-cancer agent that induces a hypoxic stress response in tumor cells to be used in conjoint therapy with a Na⁺/K⁺-ATPase inhibitor in an oral dosage form for treating a patient having a neoplastic disorder, wherein the oral dosage form comprises a total daily dose of from about 2.25 to about 7.5 mg per human individual.

222-251. (canceled)

252. A method for promoting treatment of patients having a neoplastic disorder, comprising packaging, labeling and/or marketing an anti-cancer agent that induces a hypoxic stress response in tumor cells to be used in conjoint therapy with scillaren in an oral dosage form for treating a patient having a neoplastic disorder.

253. Use of an anti-cancer agent that induces a hypoxic stress response in tumor cells in the packaging, labeling and/or marketing of a medicament, for promoting treatment of patients having a neoplastic disorder, said anti-cancer agent is administered in conjoint therapy with a Na⁺/K⁺-ATPase inhibitor in an oral dosage form, wherein the oral dosage form maintains an effective steady state serum concentration of from about 10 to about 700 ng/mL.

254-256. (canceled)

257. Use of an anti-cancer agent that induces a hypoxic stress response in tumor cells in the packaging, labeling and/or marketing of a medicament, for promoting treatment of patients having a neoplastic disorder, said anti-cancer agent is administered in conjoint therapy with a Na⁺/K⁺-ATPase inhibitor in an oral dosage form, wherein the oral dosage form comprises a total daily dose of from about 2.25 to about 7.5 mg per human individual.

258-287. (canceled)

288. Use of an anti-cancer agent that induces a hypoxic stress response in tumor cells in the packaging, labeling and/or marketing of a medicament, for promoting treatment of patients having a neoplastic disorder, said anti-cancer agent is administered in conjoint therapy with scillaren in an oral dosage form.
